(19) United States
(12) Patent Application Publication
(10) Pub. No.: US 2025/0331726 A1
(43) Pub. Date: Oct. 30, 2025

(54) FLOW-DIRECTED DEVICES FOR MEASURING PHYSIOLOGICAL DATA IN RIGHT HEART, AND METHODS AND SYSTEMS THEREOF

(71) Applicant: FANNIN PARTNERS, LLC (D/B/A FANNIN INNOVATION STUDIO), Houston, TX (US)

(72) Inventors: Reynolds M. Delgado, III, Houston, TX (US); Niloufar Jafari, Houston, TX (US); Christine Luk, Houston, TX (US); Amanda Gibbens, Houston, TX (US); Michael J. Heffernan, Katy, TX (US); Dev Chatterjee, Houston, TX (US); Atul Varadhachary, Bellaire, TX (US); Leo Linbeck, III, Houston, TX (US)

(21) Appl. No.: 18/231,172

(22) PCT Filed: Feb. 8, 2022
(86) PCT No.: PCT/US2022/015666

§ 371 Date: Aug. 7, 2023

Related U.S. Application Data
(60) Provisional application No. 63/146,927, filed on Feb. 8, 2021.

(51) Int. Cl. A61B 5/0215; A61B 5/00; A61B 5/15

(52) U.S. Cl. CPC A61B 5/0215; A61B 5/15003; A61B 5/150206; A61B 5/6853; A61B 5/6869; A61B 2560/0462; A61B 2562/0257; A61B 2562/227; A61B 2562/242

(57) ABSTRACT

An apparatus includes an elongate body including a proximal portion and a distal portion. The elongate body defining a lumen. A set of sensors are disposed on the distal portion of the elongate body. An electrical connector may be coupled to the proximal portion of the elongate body, and is coupled to the set of sensors via an electrical wire extending through the elongate body. A guide member is disposed on the distal portion near the set of sensors, the guide member transitionable between an undeployed configuration and a deployed configuration. The guide member in the deployed configuration is configured to guide movement of the distal portion of the elongate body through a vascular system of a subject using blood flow. The elongate body may have a diameter less than or equal to 8 millimeters (mm).

### CROSS-REFERENCE TO RELATED APPLICATIONS

This application claims priority to U.S. Provisional Patent Application No. 63/146,927, filed Feb. 8, 2021, titled “FLOW-DIRECTED DEVICES FOR MEASURING PHYSIOLOGICAL DATA IN RIGHT HEART, AND METHODS AND SYSTEMS THEREOF,” the disclosure of which is incorporated by reference herein.

### TECHNICAL FIELD

Systems, devices, and methods described herein relate generally to medical instruments, including catheters, and more specifically, relate to systems for introducing catheters into body cavities. In some embodiments, systems, devices, and methods described herein provide mechanisms for performing right heart catheterization through antecubital vein access using a minimal surgically sterile field.

### SUMMARY

Systems, devices, and methods described herein relate generally to mechanisms for measuring physiological data in a right heart and pulmonary arteries of a subject. In some embodiments, an apparatus includes an elongate body having a proximal portion and a distal portion. The elongate body defines a lumen. The apparatus also includes a set of sensors disposed on the distal portion of the elongate body. An electrical connector is coupled to the proximal portion of the elongate body and coupled to the set of sensors via an electrical wire extending through the elongate body. The apparatus also includes a guide member disposed on the distal portion near the set of sensors. The guide member is transitionable between an undeployed configuration and a deployed configuration. The guide member in the deployed configuration is configured to guide movement of the distal portion of the elongate body through a vascular system of a subject using blood flow. The elongate body has a diameter less than or equal to 8 millimeters (mm).

In some embodiments, a system includes a catheter and a housing. The catheter includes an elongate body having a proximal portion and a distal portion, and the elongate body defines a lumen. The catheter also includes a set of sensors disposed on the distal portion of the elongate body and a guide member disposed on the distal portion near the set of sensors. The guide member is transitionable between an undeployed configuration and a deployed configuration. The guide member in the deployed configuration is configured to guide movement of the distal portion of the elongate body through a vascular system of a subject using blood flow. The housing includes a sterile chamber configured to house a portion of the elongate body of the catheter and a connector configured to couple to a hub of an intravenous catheter such that the distal portion of the catheter can be advanced out of the sterile chamber and through a lumen of the intravenous catheter and into the vascular system.

In some embodiments, a method includes coupling a connector of a housing containing a right heart catheter to a hub of an intravenous catheter. The method also includes advancing a distal portion of the right heart catheter through a lumen of the intravenous catheter and into a peripheral vein of a subject. The method further includes deploying a guide of the right heart catheter such that the guide enables the distal portion of the right heart catheter to be carried by blood flow through a vascular system of the subject and to a pulmonary vessel of a heart of the subject. The method also includes measuring one or more physiological conditions of the subject via a set of sensors of the right heart catheter when the distal portion of the right heart catheter is disposed in the pulmonary vessel.

### BRIEF DESCRIPTION OF DRAWINGS

FIG. 1 shows an example of a Swan-Ganz catheter.

FIG. 2 shows a schematic of an example catheter for right heart catheterization (RHC), according to an embodiment.

FIG. 3A illustrates different access sites for a right heart catheterization procedure, according to an embodiment.

FIG. 3B illustrates the navigation of a right heart catheter through a heart and the different characteristic pressure waves in the different regions of the heart, according to an embodiment.

FIGS. 4A and 4B show two examples of cross-sectional views of a body of the catheter shown in FIG. 2, according to an embodiment.

FIGS. 5A and 5B show two examples of side views of a catheter for RHC, according to an embodiment.

FIG. 6 shows a schematic of an example system for RHC, according to an embodiment.

FIG. 7 shows a schematic of another example system for RHC, according to an embodiment.

FIG. 8 shows a schematic of a system including a spool to store a catheter, according to an embodiment.

FIG. 9 depicts the system shown in FIG. 8 when the system is coupled to an IV catheter, according to an embodiment.

FIGS. 10A and 10B depict undeployed and deployed states, respectively, of a guide implemented as an inflatable balloon or self-expanding gel, according to an embodiment.

FIGS. 11A and 11B depict undeployed and deployed states, respectively, of a guide implemented as a sail, according to an embodiment.

FIGS. 12A and 12B depict undeployed and deployed states, respectively, of a guide implemented as a parachute, according to an embodiment.

FIGS. 13A to 13C depict a guide implemented as a sail that can be transitioned between three configurations, according to an embodiment.

FIGS. 14A to 14C depict a guide that can be configured as a sample collection element, according to an embodiment.

FIGS. 15A and 15B depict the operation of a guide and a sample collection element, according to an embodiment.

FIG. 16 shows a schematic of a system for data acquisition and analysis associated with operating the catheters described herein, according to an embodiment.

FIG. 17 is a flowchart illustrating an example method of operating catheters described herein, according to an embodiment.

FIG. 18 is a flowchart illustrating an example method of data analytics associated with operating catheters described herein, according to an embodiment.

FIGS. 19A, 19B, and 19C depict three types of connection ports that extend from the control unit and connect to the catheter housed within a sterile casing, according to various embodiments.

FIG. 20 depicts a system including a spool and pair of feeders for storing, deploying, and/or torquing a catheter, according to an embodiment.

FIG. 21A depicts a system including a sterile casing and two feeders for storing, deploying, and/or torquing a catheter, according to an embodiment. FIG. 21B is a side cross-section view of a portion of the system of FIG. 21A taken along the line X-X shown in FIG. 21A.

FIGS. 22A and 22B depict a spool system implemented as a structure with a spring mechanism, according to an embodiment.

FIG. 23 depicts a pair of feeders coupled to a torquing assembly for advancing, retracting, and/or torquing a catheter, according to an embodiment.

FIG. 24 depicts a spool system implemented as an infinity loop structure, according to an embodiment.

FIG. 25 depicts a deployment system implemented as a control unit or user interface, according to an embodiment.

FIG. 26 depicts a spool system implemented as a cassette structure, according to an embodiment.

FIGS. 27A to 27D depict a guide implemented as an expandable balloon filled with a self-expandable structure, according to an embodiment. FIG. 27A depicts the guide in an undeployed state. FIG. 27B depicts the guide in a deployed state. FIGS. 27C and 27D depict cross-sections of the guide in the deployed state taken along the line A-A (FIG. 27C) and line B-B (FIG. 27D) as shown in FIG. 27B.

FIGS. 28A to 28D depict a guide implemented as an expandable balloon with engraved slits, according to an embodiment. FIG. 28A depicts the guide in an undeployed state. FIG. 28B depicts the guide in a deployed state. FIGS. 28C and 28D depict cross-sections of the guide in the deployed state taken along the line C-C (FIG. 28C) and line D-D (FIG. 28D) as shown in FIG. 28B.

FIGS. 29A and 29B depict undeployed (FIG. 29A) and deployed (FIG. 29B) states of a guide implemented as a self-expandable sail, according to an embodiment.

FIGS. 30A and 30B depict undeployed (FIG. 30A) and deployed (FIG. 30B) states of a guide implemented as a self-expandable sail with webbing, according to an embodiment.

FIGS. 31A to 31D depict undeployed (FIGS. 31A and 31B) and deployed (FIGS. 31C and 31D) states of a guide implemented as an expandable cup, according to an embodiment.

FIGS. 32A to 32D depict undeployed (FIG. 32A), deployed (FIG. 32B), and flipped (FIG. 32C and 32D) states of a guide implemented as an expandable cup with holes, according to an embodiment.

FIGS. 33A and 33B depict undeployed (FIG. 33A) and deployed (FIG. 33B) states of a guide implemented as an expandable cup with cut-through slits, according to an embodiment.

FIGS. 34A and 34B depict undeployed (FIG. 34A) and deployed (FIG. 34B) states of a guide implemented as an expandable cup with engraved slits, according to an embodiment.

FIGS. 35A and 35B depict undeployed (FIG. 35A) and deployed (FIG. 35B an) states of a guide implemented as an expandable cup with engraved triangles, according to an embodiment.

FIGS. 36A and 36B depict undeployed (FIG. 36A) and deployed (FIG. 36B) states of a guide implemented as an expandable cup with a closed flat surface, according to an embodiment.

### DETAILED DESCRIPTION

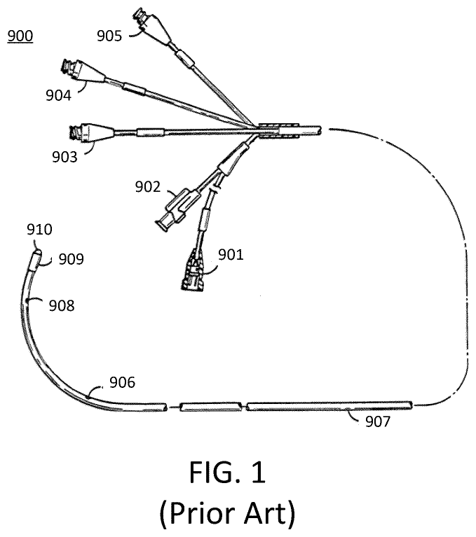

Right heart catheterization (RHC), also known as pulmonary artery catheterization, is helpful in the diagnosis, treatment, and management of various medical conditions, such as heart failure, pulmonary hypertension, heart transplants, valvular heart disease, and cardiomyopathy. Known procedures to conduct RHC can use Swan-Ganz catheters. Suitable examples of such catheters are described in EP Patent Application Publication No. EP 0303756, published Feb. 22, 1989, titled “Thermodilution and pressure transducer balloon catheter,” EP Patent Application Publication No. 0363117, published Apr. 11, 1990, titled “A position-monitoring flow-directed catheter and method,” and U.S. Pat. No. 3,995,623, published Dec. 7, 1976, titled “Multipurpose flow-directed catheter,” each of which are incorporated herein by reference. FIG. 1, reproduced from EP Patent Application Publication No. 0363117, shows an example Swan-Ganz catheter 900. The Swan-Ganz catheter 900 includes multiple proximal end connector tubes 901 to 905 (also referred to as connectors 901 to 905). The connector 901 is configured for connection to an inflation device (e.g., a balloon 909) to facilitate the motion of the catheter 900 within the vein of a patient. The inflation medium in the inflation device can include, for example, bacteria-filtered carbon dioxide. The connector 902 is configured to couple a thermistor located at the distal end of the catheter 900.

The connector 903 is configured to connect to a pressure measurement device (also referred to as a pressure transducer), which can be disposed at a distal port opening 910. The pressure measurement device is configured to record pulmonary arterial pressures when the balloon is deflated and pulmonary wedge pressures when the balloon is inflated and in the pulmonary artery wedge position.

The connector 904 is coupled to an injectate lumen (i.e., the lumen to inject a material into the vein of the patient), which can be used for multiple purposes, such as right atrium pressure monitoring, blood sampling, injection and/or infusion of liquids for therapeutic or diagnostic purposes, and right atrial pacing by insertion of an electrode probe through a port opening 907. The connector 905 is configured for connection of a position-monitoring device, which can be used to determine the position of the distal tip of the catheter (e.g., the position of a port 906).

Known catheters, such as the Swan-Ganz catheter 900, have several drawbacks. First, use of these catheters usually requires a large sterile field and multiple sterile connectors (e.g., at least three). In addition, the accompanying monitoring equipment and programs are typically available in an intensive care unit (ICU) or a catheterization laboratory (cath lab) and not available for outpatient use, other hospital settings, or clinical settings. For example, the pressure transducer and oxygen saturation (SvO₂) machines in these catheters generally involve complicated and cumbersome calibration and/or certification. Furthermore, the dimensions of known catheters are very large (e.g., 6 French, or 2 mm for pressures, and 7 French, or 2.33 mm, for cardiac output (CO)). Therefore, these catheters are typically delivered via larger access sites and veins that require anesthesia.

Apparatus, systems, and methods descried herein employ miniaturized catheters (e.g., catheters with smaller lateral profiles) that allow in-office RHC. These catheters require a small sterile field because they enclose portions of the system (e.g., including the catheter body defining one or more lumens) in a sterile housing (e.g., a sterile spool). In some embodiments, the catheters include a small number of connectors that can be compatible with small profile devices (e.g., 18-gauge intravenous (IV) catheters), and each connector can include a wire or a combination of a wire and a lumen coming from the spool. A sensor (e.g., solid state sensor) is used in the catheters to monitor pressure and/or other physiological data, and the acquired data is transmitted, processed, and displayed on one or more external computer systems. In some embodiments, the flow or movement of the catheters is controlled by a deployable guide (e.g., a sail, a balloon, etc.). These features (individually or in combination) can enable use of the catheters in outpatient and other clinical settings without requiring a large sterile field and/or anesthesia.

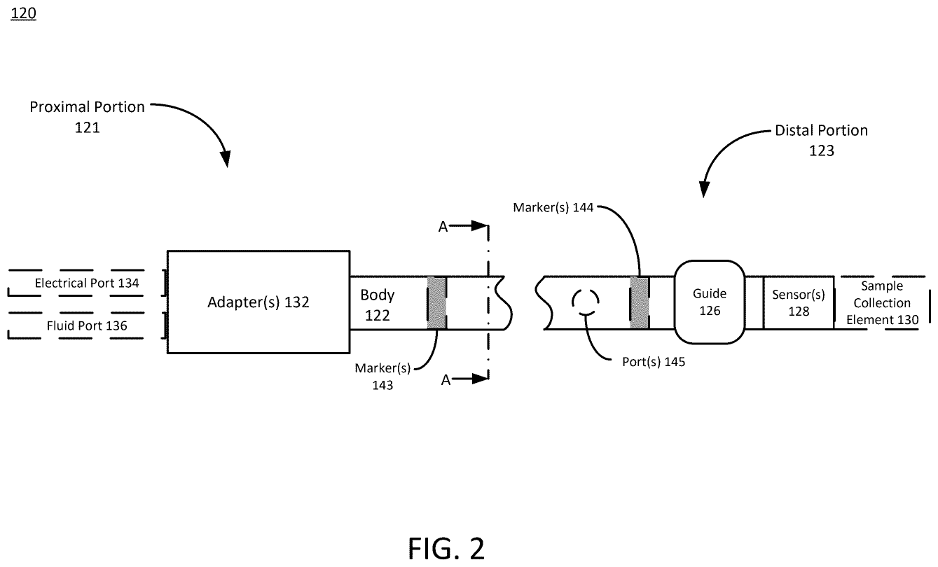

FIG. 2 shows a schematic of an example catheter 120, e.g., that can be used in an in-office setting for right heart catheterization (RHC), according to embodiments described herein. The catheter 120 includes a proximal portion 121 and a distal portion 123 connected by a body 122. In some embodiments, during use of the catheter 120, the distal portion 123 and at least a portion of the body 122 are configured to be inserted into a vein of a patient (e.g., antecubital vein) and navigated through peripheral vasculature to a right side of the heart of a patient, and the proximal portion 121 is configured to be disposed outside the body of the patient. As further described below, the distal portion 123 and the portion of the body 122 to be inserted into the body of the patient can be stored in a sterile environment prior to use.

The proximal portion 121 of the catheter 120 can include one or more adapters 132 and optionally include one or more of an electrical port 134 and a fluid port 136. The adapter(s) 132 are configured to securely couple the body 122 of the catheter 120 with external ports, such as the electrical port 134 and the fluid port 136.

In some embodiments, the electrical port 134 is configured to receive a wire or a cable that is used to connect sensor(s) 128 with external components (e.g., power supply, data processing unit, etc.). In some embodiments, the electrical port 134 is configured to be coupled to a connector (e.g., electrical connector) that facilitates further connection with a wire or cable to a remote compute device.

In some embodiments, the fluid port 136 is configured to facilitate the operation of (e.g., deployment of) the guide 126. For example, the guide 126 can be implemented as or include a balloon, and the fluid port 136 can be configured to deliver a fluid (e.g., liquid, gas) to inflate the balloon. In some embodiments, the fluid can include air, helium, or a mixture of helium and oxygen. In some embodiments, the fluid can be stored in a pre-attached syringe (not shown in FIG. 1) and motorized pump can be used to deliver a controlled amount of gas into the balloon. In another example, the fluid port 136 can be used to deliver a material that forms the guide 126 (e.g., an expanding foam), which can subsequently degrade or be removed from the body after deployment. In some embodiments, the catheter 120 may not include a fluid port 126. In such embodiments, the catheter 120 can include a guide 126 that can be implemented as or include a mechanically and/or electrically operated structure (e.g., an umbrella structure). The catheter 120 can be configured to receive a wire to mechanically and/or electrically control the opening and closing of the structure, or the catheter 120 can include a retaining element with a release mechanism that can release to deploy the structure.

In some embodiments, the guide 126 includes a bi-metallic strip or coil, which can be transitioned between the undeployed state and the deployed state via temperature change. For example, an increase in temperature can cause the bi-metallic strip or coil to bend or deform, thereby changing its volume. In some embodiments, the guide 126 includes a shape memory material, an electroactive polymer, or an alloy. In some embodiments, the guide 126 may include a bilayer such that the properties of one layer included in the bilayer are different from the other layer included in the bilayer (e.g., different materials, different thicknesses, etc.) which allows the first layer to have a different amount of expansion than the second layer due to exposure to temperature, moisture, etc. The different expansion rates may cause the guide to bend to be moved from the undeployed to the deployed state.

In some embodiments, the catheter 120 includes two ports (e.g., the electrical port 134 and the fluid port 136) and does not include any other port. In some embodiments, the catheter 120 can include one port (e.g., the electrical port 134). In some embodiments, the catheter 120 can include more than two ports. It can be desirable for the catheter 120 to include a small number of ports and/or accompanying lumens or connectors such that the catheter 120 has a smaller diameter or profile.

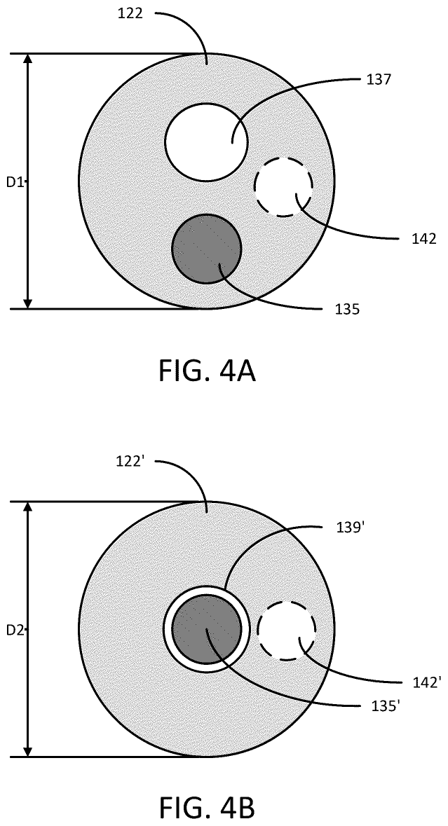

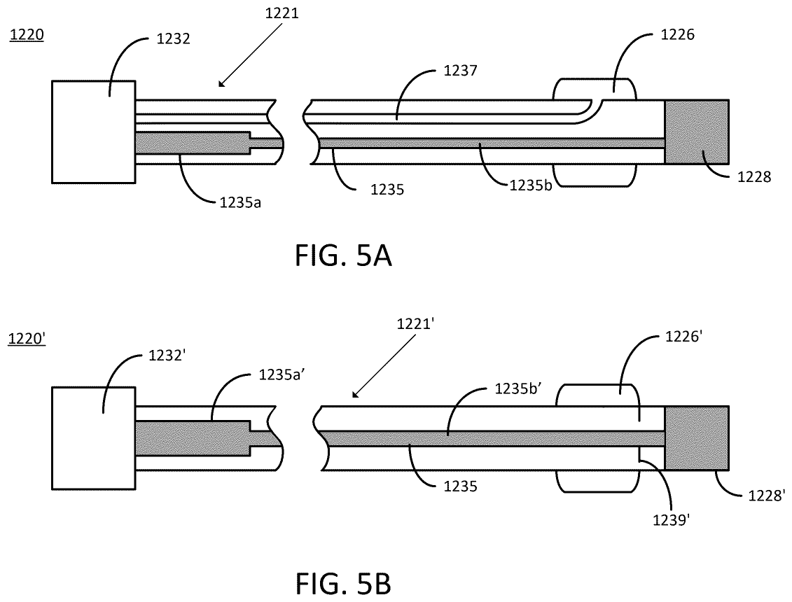

The body 122 of the catheter 120 can extend distally from the adapter(s) 132. A portion of the body 122, adapter(s) 132, and/or port(s) 134 and 136 of the catheter 120 can be partially located within a housing (as further described with respect to FIG. 5), with the distal portion 123 capable of being extended into and navigated through peripheral vasculature to the right side of the heart. In some embodiments, the body 122 can define at least one lumen. In some embodiments, the body 122 defines a first lumen for electrical connection (e.g., for connection with sensor(s) 128) and a second lumen for delivering a fluid (e.g., for deployment of the guide 126). More details about the body 122 and the lumen(s) defined by the body 122 are provided below (e.g., with reference to FIGS. 4A and 4B).

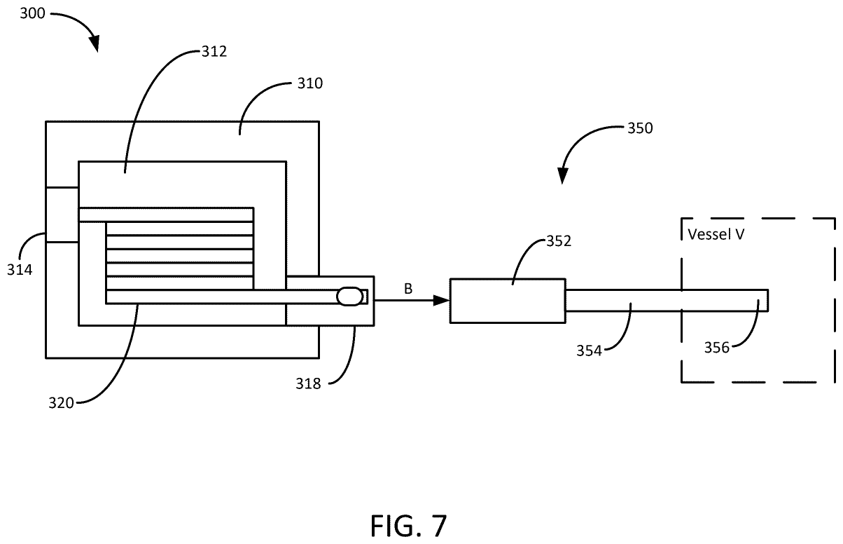

In some embodiments, the length of the body 122 of the catheter 120 can be about 20 cm or longer (e.g., about 20 cm, about 25 cm, about 30 cm, about 35 cm, about 40 cm, about 50 cm, or longer, including any values and subranges in between). In some embodiments, the diameter of the body 122 can be about 8 mm or less (e.g., about 8 mm, about 7 mm, about 6 mm, about 5 mm, about 4 mm, about 3 mm, about 2 mm, about 1.33 mm, about 1 mm, or less, including any values and subranges in between). In some embodiments, the body of the 122 can be inserted through an IV catheter. The IV catheter needle size can range from about 15-gauge to about 18-gauge. Depending on the access site (e.g., antecubital fossa veins, or femoral vein), or other applications, the IV catheter needle can be from about 25-gauge to about 10-gauge, including any sizes and subranges in between (e.g., from the largest to the smallest diameter including 10-gauge, 11-gauge, 12-gauge, 13-gauge, 14-gauge, 15-gauge, 16-gauge, 17-gauge, 18-gauge, 19-gauge, 20-gauge, 21-gauge, 22-gauge, 23-gauge, 24-gauge, and 25-gauge). In some embodiments, the body 122 of the catheter 120 can be stored in a housing, e.g., using a spool or a reel so as to reduce the sterile field required to use the catheter 120 (as further described with reference to FIGS. 7 to 9 and 19A to 26 below).

The distal portion 123 of the catheter 120 includes a guide 126, one or more sensor(s) 128, and optionally one or more port(s) 145 and/or a sample collection element 130. The guide 126 is transitionable (or switchable) between a first, undeployed configuration and a second, deployed configuration. In the deployed configuration, the guide 126 is configured to guide the movement of the distal portion 123 of the catheter 120 through the vascular system of the patient using the blood flow (e.g., by catching and being carried by the blood flow). In some embodiments, the guide 126 includes a balloon, which can be transitioned into the deployed configuration by inflating the balloon with liquid or gas (e.g., via the fluid port 136 and a lumen connected to the fluid port 136). In some embodiments, the guide 126 includes a flow-directing sail, which can be transitioned into the deployed configuration by opening an umbrella structure. In some embodiments, the guide 126 can be configured to automatically transition into the deployed configuration in response to the release of a retaining element (e.g., wires, sheath, etc.). In some embodiments, the guide 126 can be configured to transition between the undeployed and deployed configurations based on manual and/or automatic control (including, for example, mechanically and/or electrically controlled mechanisms), e.g., such that the flow speed and/or movement of the catheter 120 can be controlled (e.g., adjusted). In some embodiments, systems described herein can include a deployment mechanism configured to transition the guide 126 from the undeployed configuration to the deployed configuration. For example, the deployment mechanism can include a slider that is coupled via a shaft to the guide 126, and the slider is configured to move to slide the shaft to transition the guide 126 from the undeployed configuration to the deployed configuration. In some embodiments, the shaft can be slidably disposed within a lumen defined by the body 122. In some embodiments, the shaft can be made of nitinol and an electrical current can be passed through the shaft to cause contraction of the shaft, thereby controlling the deployment of the sail.

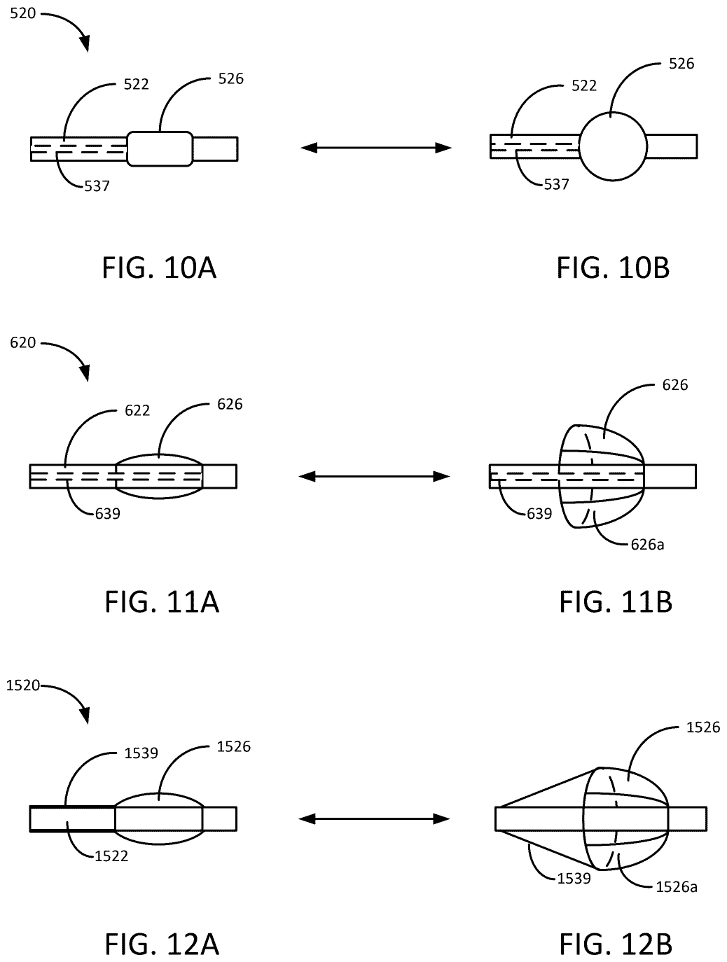

In some embodiments, the guide 126 can be configured to transition into multiple configurations, e.g., an undeployed configuration, a fully deployed configuration, and one or more intermediate configurations. For example, the guide 126 can be transitioned from an undeployed configuration into a first deployed configuration to guide the catheter 120 through the vasculature to a target site in the heart, and then be transitioned to a second deployed configuration to occlude a vessel (e.g., a branch of a pulmonary artery) for obtaining certain physiological measurements (e.g., wedge pressure), as further described below. More details about the guide 126, including different embodiments of a guide 126, are provided below with reference to, for example, FIGS. 10A to 14C and FIGS. 27A to 36B.

The catheter 120 also optionally includes marker(s) 143 and 144 disposed on the body 122. In some embodiments, the maker(s) 143 and 144 can be used to determine the length of the body 122 within the vascular system of the patient and accordingly derive the location of the distal portion 123 of the catheter 120. In some embodiments, the catheter 120 includes two types of markers 143 and 144. A first set of one or more markers 143 can be disposed more proximal on the catheter 120 such that an operator can read the markers 143 to determine the progress of the catheterization. A second set of one or more markers 144 can be disposed in the distal portion 123 of the catheter 120 and therefore are within the vascular system of the patient during catheterization. In some embodiments, the second set of markers 144 can include one or more active trackers (e.g., emitters) configured to emit a signal (e.g., radio frequency or RF signal, optical signal, etc.) so as to indicate the location of the distal portion 123 of the catheter 120. In some embodiments, the second set of markers 144 can include one or more passive trackers (e.g., solenoid microcoils) that are configured to respond to an externally applied signal (e.g., RF illumination) so as to indicate the location of the distal portion 123 of the catheter 120. Any other appropriate markers can also be used. In some embodiments, the catheter 120 includes one set of markers (e.g., either 143 or 144). In some embodiments, the catheter 120 can include additional markers located on the proximal portion, such as, for example, a scale disposed on the outer surface of the body 122, labels or tags indicating information regarding the catheter 120, etc.

The sensor(s) 128 are configured to measure physiological information of the subject, e.g., at the distal portion 123 of the catheter. In some embodiments, the sensor(s) 128 can be configured to measure the pressure of the environment surrounding the distal portion 123 of the catheter (e.g., pulmonary artery pressure, pulmonary capillary wedge pressure (PCWP), right ventricular pressure, right atrial pressure, etc.). In some embodiments, the sensor(s) 128 can include one or more solid state sensors. In some embodiments, the sensor(s) 128 include one or more fiber optical pressure sensors. For example, the fiber optical pressure sensors can include a Fabry-Perot etalon, which has one or more transmission peaks (or valleys) depending on the distance between two reflectors in the etalon (also referred to as cavity length). The pressure of the surrounding environment can change the cavity length and therefore change the location of the transmission peaks (or valleys) in the frequency domain. Accordingly, by monitoring the location of the transmission peaks, one can estimate the surrounding pressure. In some embodiments, the sensor(s) 128 include one or more metal diaphragms with resistive strain gauges. The surrounding pressure can change the resistance (or conductivity) of the strain gauges and accordingly the amplitude of electrical current running through the strain gauges. Therefore, the reading of the electrical current can be used to estimate the surrounding pressure. In some embodiments, the sensor(s) 128 can be configured to measure one or more of pressure, oxygen concentration, electrocardiogram or heart rate, temperature, etc. In some embodiments, the sensor(s) 128 can include pressure sensors, image or light sensors, temperature sensors, gas sensors, magnetic sensors, etc. In some embodiments, the sensor(s) 128 can be configured to wirelessly transmit data indicative of one or more measured physiological conditions to a communications component or element or to a compute device (e.g., a local control unit, or a remote compute device, or a deployment system, as further described herein).

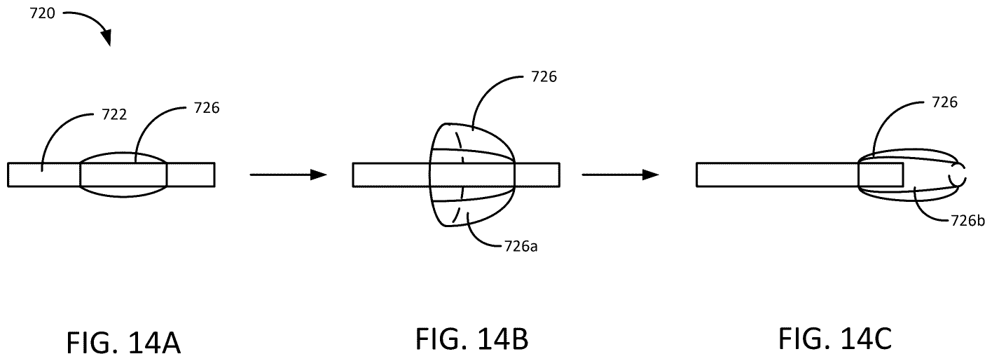

In some embodiments, the catheter 120 optionally includes a sample collection element 130. The sample collection element 130 in the catheter 120 is configured to collect one or more samples (e.g., blood or tissue samples) during catheterization. For example, the sample collection element 130 can be configured to collect a blood sample for further analysis, such as measurement of gas concentrations, complete blood count (CBC), chemistry panels, nutrient tests, etc. In some embodiments, the catheter 120 can include sensor(s) (e.g., sensor(s) 128 or other sensors coupled to the catheter 120), such as oxygen or other blood gas sensors, so as to allow real-time measurement of blood properties during the catheterization process. In some embodiments, the sample collection element 130 can be implemented as and/or with a guide 126. For example, a guide 126 can be configured to transition into a first configuration to guide the catheter 120 to a desired location and be configured to transition from the first configuration to a second configuration to capture a sample of blood. Further details regarding such an embodiment are provided with reference to FIGS. 14A to 14C and FIGS. 27A to 36B.

In some embodiments, the catheter 120 optionally includes port(s) 145. The port(s) 145 can be configured to couple to a fluid source (e.g., via an internal lumen of the catheter 120) to deliver a fluid into the vasculature or heart. For example, the port(s) 145 can deliver fluids containing therapeutic agents, pharmaceutical agents, contrast agents (e.g., for imagining and location determination), etc.

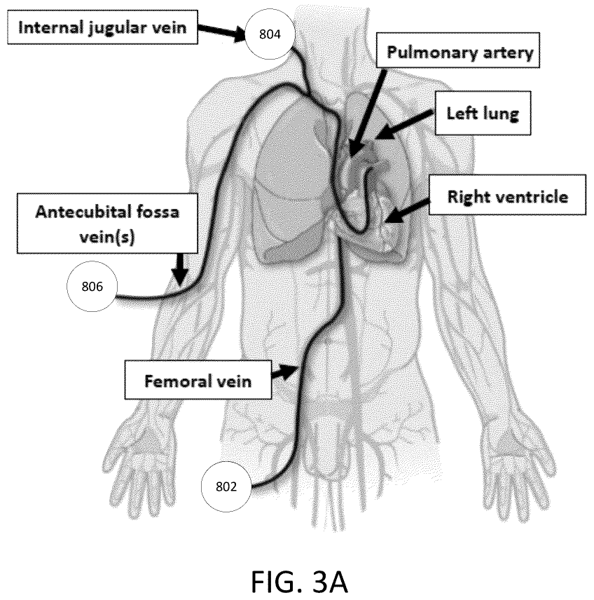

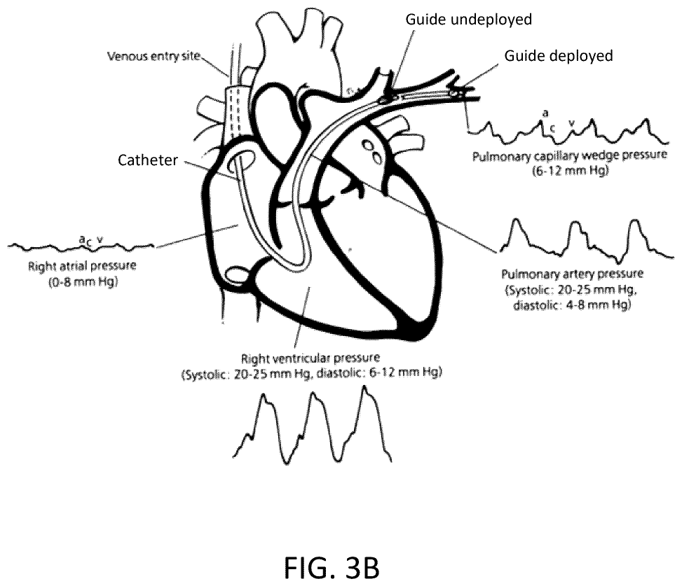

FIG. 3A illustrates different access sites for a right heart catheterization procedure, according to an embodiment. Catheters described herein (e.g., the catheter 120 shown in FIG. 2) can be inserted into a vascular system of a patient via access sites 802, 804, and 806. A first access site 802 provides access to the heart of the patient via the femoral vein. A second access site 804 provides access to the heart of the patient via the internal jugular vein. A third access site 806 provides access to the heart of the patient via a peripheral vein of an arm (e.g., the antecubital fossa veins, the brachial vein). The third access site 806 can involve navigating through vasculature with significantly reduced dimensions as compared to the first and second access sites 802, 804. In contrast, conventional catheters (e.g., Swan-Ganz catheters) are usually inserted into the vascular system of the patient via the first access site 802 (femoral vein) and the second access site 804 (internal jugular vein). As described above, such conventional catheters may not be capable of being inserted through the third access site 806, e.g., through the antecubital fossa veins, without anesthesia due to the size of those catheters.

Different characteristic pressure waves can be associated with different regions of the heart as catheter devices described herein navigate through the heart, as described in the article “Pulmonary Artery Catheter,” authored by Umesh K. Gidwana et al., published 2013, and available at https://www.cardiology.theclinics.com/article/S0733-8651(13)00071-4/pdf. FIG. 3B illustrates the different characteristic pressure waves in the different regions of a heart (e.g., right atrium, right ventricle, pulmonary artery, etc.), as described in the article. In particular, after a catheter (e.g., the catheter 120 shown in FIG. 2) is inserted into one of the access sites 802, 804, or 806, the catheter can be guided (e.g., via the guide of the catheter (e.g., guide 126) and blood flow) to the right heart of the patient.

As the catheter advances near or within the heart, the location of the distal portion of the catheter (e.g., distal portion 123) can be determined by characteristic pressure wave forms, e.g., acquired by a pressure sensor disposed at the distal portion of the catheter (e.g., sensor(s) 128). More specifically, when the catheter is in the right atrium (RA), the RA pressure wave forms (labelled as right atrial pressure) are characterized by two positive waves. The first positive wave (labelled as an “a” in FIG. 3B) occurs during RA systole and the second positive wave (labelled as a “v” in FIG. 3B) occurs during the end of right ventricular (RV) systole. There are also two negative waves. The first negative wave (also referred to as the “x” descent) is caused by atrial relaxation and the second negative wave (also referred to as the “y” descent) occurs during RV rapid filling. An additional positive wave, referred to as the “c” wave, may also be observed during the “x” descent. Without being bound by any particular theory or mode of operation, the “c” wave can result from doming of the tricuspid valve into RA during RV isovolumic systole.

With further advancement, the distal portion of the catheter crosses the tricuspid valve and the right ventricular (RV) pressure wave form can be observed. The RV pressure waveform is characterized by a sharp upstroke during the isovolumic phase of systole. During the ejection phase, the rise in pressure is much slower. During the isovolumic relaxation phase, the RV waveform has a sharp down stroke. Because of the tricuspid valve, the RV systolic pressure is generally higher than the RA pressure. During diastole, a rapid filling wave, diastasis, and atrial filling waves can be recognized. During the rapid filling phase, RV diastolic pressure can increase with inflow of blood from the RA. During the phase of diastasis, no further rise in RV pressure occurs because inflow from the RA ceases due to the lack of a pressure gradient between the two chambers. The atrial filling wave is related to RA contraction at the end of diastole.

After the right ventricle, the catheter crosses the pulmonic valve and enters the pulmonary artery (PA), recognized by a characteristic pressure wave form referred to as the pulmonary artery pressure (i.e., PA pressure). The PA pressure waveform is characterized by a sharp upstroke and a down stroke interrupted by the dicrotic notch and dicrotic wave. Further advancement of the catheter brings the catheter into a branch vessel from the PA. When the guide is set to occlude the branch artery, the catheter can measure a PCWP wave form.

FIGS. 4A and 4B show examples of cross-sectional views of the body 122 of the catheter 120, along line A-A shown in FIG. 2, according to embodiments described herein. FIG. 4A depicts that the body 122 defines a fluid lumen 137, which can be used, e.g., for delivering a fluid to inflate the guide 126 that is implemented as a balloon. The fluid lumen 137 can be operatively coupled to the fluid port 136 during use. The body 122 also includes an electrical wire 135, which can be used for connecting one or more sensor(s) 128 to the electrical port 134, which in turn can connect to a compute device (e.g., control/communications unit) for viewing and/or analyzing data collected by the one or more sensor(s) 128. Optionally, the body 122 can include one or more additional lumen(s) 142, which can be used, e.g., for conveying fluids (e.g., therapeutic agents) and/or containing additional instruments/components. For example, additional sensors (e.g., blood oxygen sensors) can be disposed at the distal end 123 of the catheter 120 and connected to devices at the proximal end 121 of the catheter 120 via the lumen 142. As another example, a therapeutic agent can be conveyed via lumen 142 to a port (e.g., port 145) for delivery into the vasculature.

FIG. 4B provides a different example cross-sectional view of a body 122′ of a catheter. The body 122′ can include a lumen 139′ in which a shaft 135′ is slidably disposed. The shaft 135′ can be slid longitudinally along a length of the body 122′ to, for example, deploy or close a guide 126 (e.g., implemented as a sail or other mechanically actuated guide). In some embodiments, the shaft 135′ can also be configured to function as an electrical connection. For example, the shaft 135′ can be configured for connecting one or more sensor(s) 128 to the electrical port 134 for viewing and/or analyzing data collected by the one or more sensor(s) 128. As another example, the shaft 135′ can be used to deliver an electrical signal to control the operation (e.g., deployment, retraction, etc.) of the guide 126. In these embodiments, the shaft 135′ can include a conductive material, such as metal, carbon, and conductive oxide, among others. In some embodiments, the shaft 135′ can include a lightweight non-conductive core coated with a conductive material. Optionally, the body 122′ can include one or more additional lumen(s) 142′, which can be used, e.g., for conveying fluids and/or containing additional instruments/components. For example, additional sensors (e.g., blood oxygen sensors) can be disposed at the distal end 123 of the catheter 120 and connected to devices at the proximal end 121 of the catheter 120 via the lumen 142′. As another example, a therapeutic agent can be conveyed via lumen 142′ to a port (e.g., port 145) for delivery into the vasculature.

Body 122 and body 122′ depicted in FIGS. 4A and 4B can have diameters D1 and D2 that are sufficiently small to be inserted through a lumen of a standard, e.g., 18-gauge, IV catheter. In some embodiments, the diameters D1 and D2 are about 8 mm or less (e.g., about 8 mm, about 7 mm, about 6 mm, about 5 mm, about 4 mm, about 3 mm, about 2 mm, about 1.33 mm, about 1 mm, or less, including any values and subranges in between). In some embodiments, the diameter D2 of the body 122′ can be less than the diameter D1 of the body 122, e.g., due to body 122′ containing less lumens and/or instruments.

Catheter devices, as described herein, can include shafts and/or lumens that vary in diameter. For example, FIGS. 5A and 5B show two examples of a side view of catheters 1220 and 1220′, with shafts that vary in diameter. Referring to FIG. 5A, the catheter 1220 includes an adapter 1232 on the proximal end and a sensor 1228 on the distal end. The proximal end and the distal end are connected by a body 1221, which defines a lumen 1237. The body 1221 further includes a shaft 1235 extending along its length (e.g., from the adapter 1232 to a distal end of the catheter 1220). The shaft 1235 includes two sections. In some embodiments, a first section 1235a of the shaft 1235 closer to a proximal end of the catheter 1220 can be configured to stay outside a patient during a catheterization procedure, while a second section 1235b of the shaft 1235 closer to the distal end of the catheter 1220 can be configured to be inserted into the vascular system of the patient during the catheterization procedure. The diameter of the second section 1235b (also referred to as the second diameter) can be less than the diameter of the first section 1235a (also referred to as the first diameter) such that the body 1221 of the catheter 1220 can have a smaller profile and/or greater flexibility for facilitating the navigation of the distal end within the vascular system of the patient. In some embodiments, the ratio of the second diameter to the first diameter is about 0.8 or less (e.g., about 0.8, about 0.7, about 0.6, about 0.5, or less, including any values and subranges in between). In addition, the diameter of the first section 1235a can be greater to facilitate and/or increase durability of connections between the shaft and one or more other components (e.g., adapter 1232 and/or an electrical port).

The shaft 1235 can be connected to the sensor 1228. In some embodiments, the shaft 1235 can be implemented as a wire or cable for powering or controlling the sensor 1228 and/or transmitting data collected by the sensor 1228 to a processing unit (not shown in FIG. 5A). For example, the wire or cable can be configured to send one or more control signals to the sensor 1228 so as to control the operation of the sensor 1228 (e.g., start/stop collecting data, change data acquisition rate, change data type, etc.). The wire or cable can also be configured to receive signals from the sensor 1228 of data collected by the sensor 1228.

The lumen 1237 can be a fluid lumen that is connected to a guide 1226. In some embodiments, the lumen 1237 is configured to provide a fluid (e.g., a liquid or gas) to the guide 1226 so as to deploy the guide 1226 (e.g., a balloon). In some embodiments, the second lumen 1237 can be configured to drain fluid from the guide 1226 so as to retract (e.g., undeploy) the guide 1226.

Referring to FIG. 5B, the catheter 1220′ includes an adapter 1232′ disposed on the proximal end. A guide 1226′ and a sensor 1228′ are disposed on the distal end. The proximal end and the distal end are connected by a body 1221′, which includes a shaft 1235′. The shaft 1235′ includes a first section 1235a′ closer to the proximal end and a second section 1235b′ closer to the distal end. The diameter of the first section 1235a′ can be greater than the diameter of the second section 1235b′.

The shaft 1235′ can be configured for multiple purposes. For example, the shaft 1235′ can be coupled to the sensor 1228′ and be configured to power the sensor 1228′, collect data acquired by the sensor 1228′, and/or send control signals to the sensor 1228′ (similar to shaft 1235, as described above). Additionally, or alternatively, the shaft 1235′ can be slidably disposed within the body 1221′ of the catheter 1220 (e.g., within a lumen) and be coupled to the guide 1226′, e.g., via a connector 1239′. The shaft 1235′ can be slid longitudinally along a length of the body 1221′ to, for example, deploy or close the guide 1226′ (e.g., implemented as a sail or other mechanically actuated guide). In some embodiments, the shaft 1235′ can be configured to electrically control the deployment or closing of the guide 1226′. In these embodiments, the shaft can include a rigid, conductive material (e.g., metal, carbon, etc.).

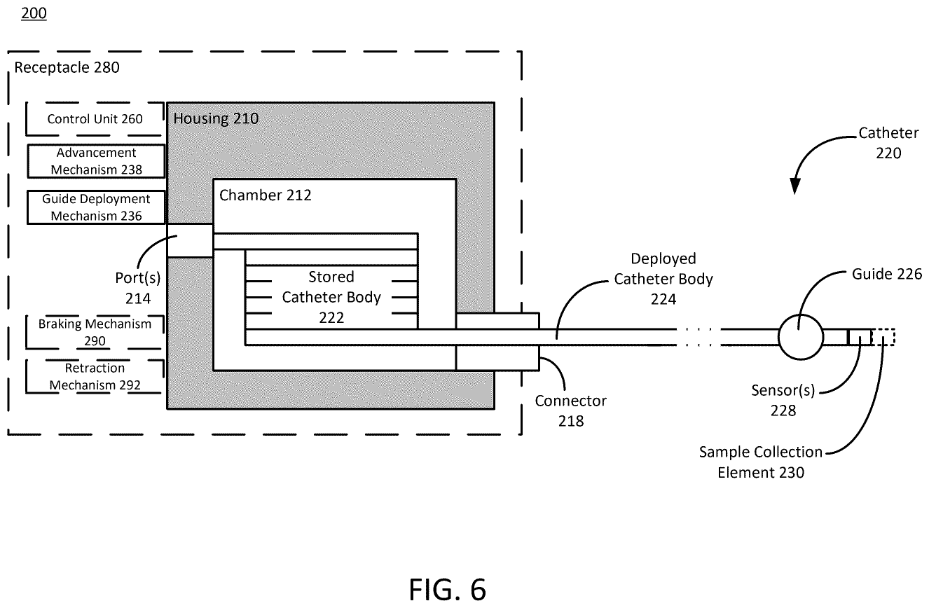

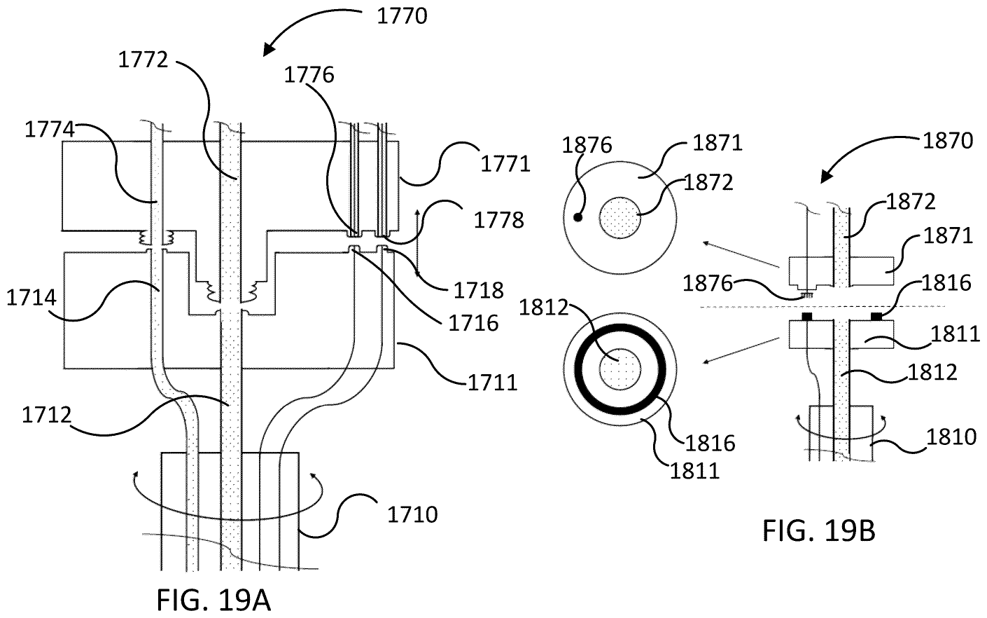

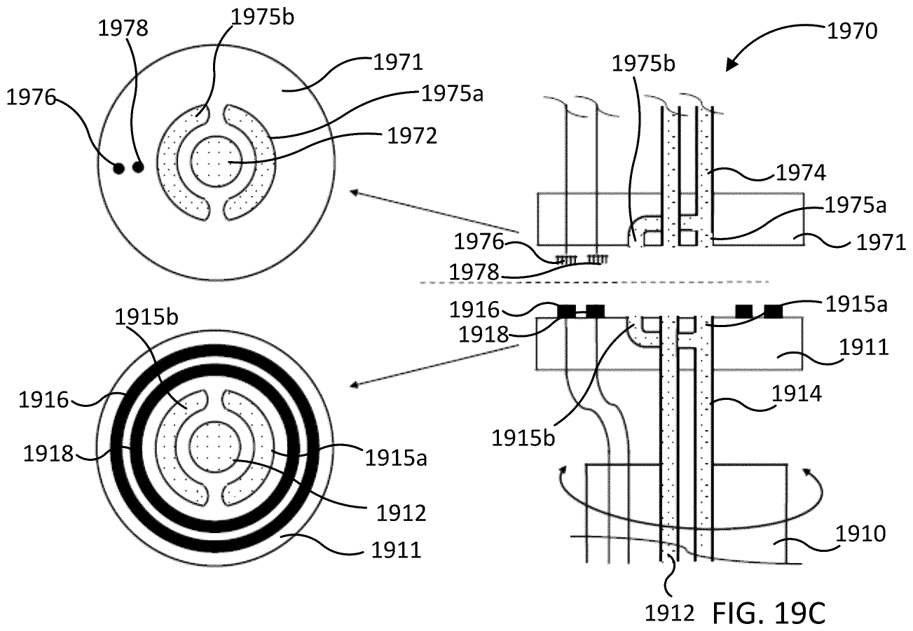

FIG. 6 shows a schematic of an example system 200 for right heart catheterization, according to an embodiment. The system 200 includes a housing 210, e.g., for containing a catheter 220 within a sterile environment (e.g., confining the catheter 220 to a limited sterile field). The catheter 220 can include components that are structurally and/or functionally similar to the catheter 120 depicted in FIG. 2. The housing 210 can define a chamber 212 for containing a portion of a catheter body 222 (also referred to as the stored catheter body 222). In some embodiments, the catheter body 222 can be wound around a spool or reel to increase the compactness of the system 200 and enable storage and/or deployment of the catheter body 222, 224 without tangling or damaging the catheter 220. More examples and details about the storage of the catheter body 222 are provided below with reference to FIGS. 19A to 26.

The housing 210 can include one or more openings or ports 214 for enabling connections to and from the catheter 220, e.g., for connections to remote device(s), fluid source(s), etc. The housing 210 also includes a connector 218 that can be connected to a hub of an IV catheter (e.g., a Luer connector) to establish a sterile connection between the housing 210 and the IV catheter. When the housing 210 is connected to the IV catheter, a portion 224 of the catheter 220 can extend out through an opening of this connector 218 and into the IV catheter, as further described with reference to FIG. 7 below. Similar to the catheter depicted in FIG. 2, the catheter 220 can include a guide 226, sensor(s) 228, and optionally a sample collection element 230.

The system 200 includes a guide deployment mechanism 236 operatively coupled to the catheter 220. The guide deployment mechanism 236 is configured to control the operation of the guide 226. In some embodiments, the guide deployment mechanism 236 includes a pump configured to deliver fluid into the guide 226 so as to deploy the guide 226. In these embodiments, the guide 226 can include a balloon. In some embodiments, the guide deployment mechanism 236 can be configured to pump or release a substance or material (e.g., an expanding foam) that can form the guide 226. In some embodiments, the guide deployment mechanism 236 can include a drive mechanism (e.g., spring, motor, etc.) configured to slide or advance a shaft, which in turn is coupled to the guide 226 (e.g., a sail or other mechanically deployed guide). Sliding the shaft back and forth along the length of the deployed catheter body 224 can deploy/undeploy the guide 226. In some embodiments, the guide deployment mechanism 236 can include a retaining element that can be released to deploy the guide 226.

The system 200 includes an advancement mechanism 238 operatively coupled to the catheter 220. The advancement mechanism 238 is configured to control the deployment of the catheter 220. In some embodiments, the advancement mechanism 238 includes a drive mechanism (e.g., a stepper motor, servo motor, or other device) to rotate a spool so as to unwind the catheter 220, allowing advancement of the catheter 220 within the vascular system of the patient. In some embodiments, the system 200 can include a sensor to read markers on the catheter (e.g., similar to markers 143 and/or 144 shown in FIG. 2) and determine the length of the portion of the catheter 220 that has been deployed (also referred to as the deployed catheter body 224). In some embodiments, the advancement mechanism 238 can be coupled to such a sensor and be configured to deploy a predetermined amount of deployed catheter body 224. Once the predetermined amount is reached, the advancement mechanism 238 is configured to stop deployment of the catheter 220. Alternatively, or additionally, the advancement mechanism 238 can be coupled to a compute device (e.g., control unit 260), which can monitor a location of a distal end of the catheter 220 (e.g., based on pressure or other sensor readings) and control the operation of the advancement mechanism 238.

In some embodiments, the system 200 includes an optional braking mechanism 290, which can be configured to slow down or stop the deployment of the catheter body 222, 224. In some embodiments, the optional braking mechanism 290 can be combined with the advancement mechanism 238 into a single component.

In some embodiments, the system 200 includes an optional retraction mechanism 292, which can be configured to retract or withdraw the catheter 220, e.g., from the vascular system of the patient. In some embodiments, the retraction mechanism 292 can be combined with the advancement mechanism 238 into a single component. For example, the advancement mechanism 238 can include a stepper motor configured to rotate the spool about which the stored catheter body 222 is wound. The stepper motor can rotate the spool in one direction (e.g., clockwise) to deploy the stored catheter body 222 and in another direction (e.g., counterclockwise) to retract the deployed catheter body 224. In some embodiments, the advancement mechanism 238, braking mechanism 290, and retraction mechanism 292 can operate to control the movement of the deployed catheter body 224 through the vasculature to the heart. For example, when the deployed catheter body 224 is off course or caught (e.g., on a vessel wall), one or more of the advancement mechanism(s) 238, braking mechanism 290, and retraction mechanism 292 can move the deployed catheter body 224 to set it back on course to the heart.

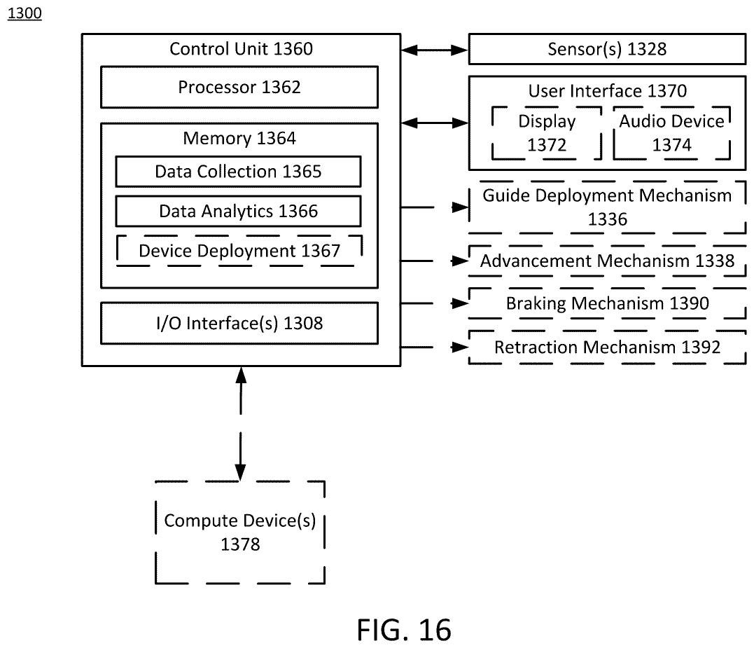

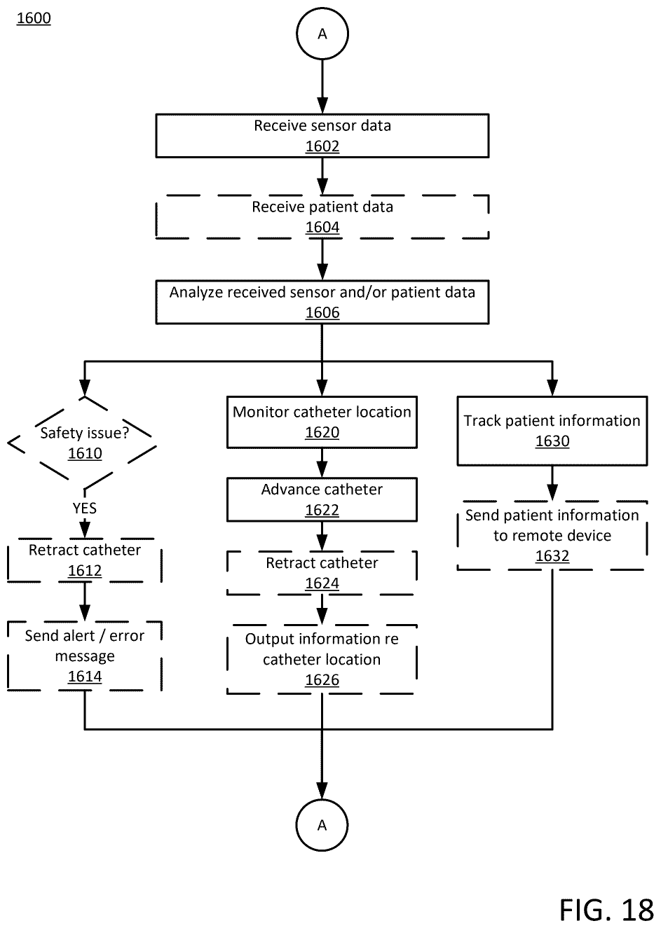

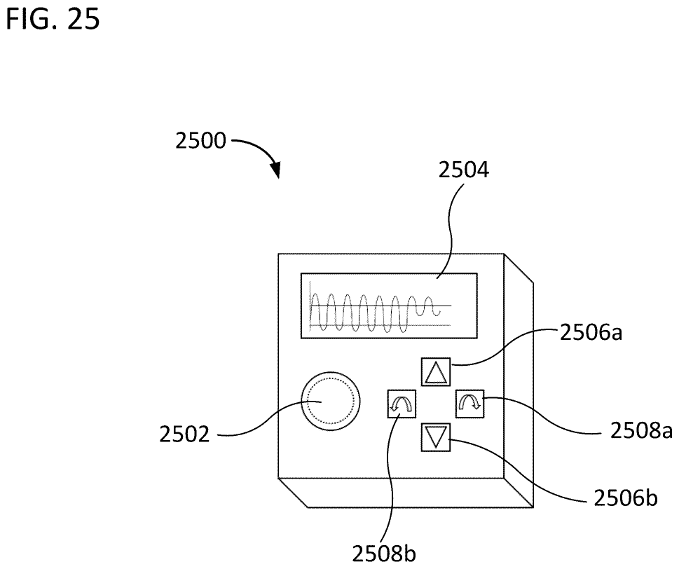

In some embodiments, the system 200 includes an optional control unit 260, which can be configured to control the operation of one or more components in the system 200 and/or receive data from or communicate data to one or more components of the system. In some embodiments, the control unit 260 can be a processor. Suitable examples of processors can include a general-purpose processor, a Field Programmable Gate Array (FPGA), an Application Specific Integrated Circuit (ASIC), a Digital Signal Processor (DSP), and/or the like. In some embodiments, the control unit 260 is configured to control the guide deployment mechanism 236 to deploy or undeploy the guide 226. In some embodiments, the control unit 260 is configured to control the operation of one or more of the advancement mechanism(s) 238, braking mechanism 290, and retraction mechanism 292 so as to control advancement of the catheter 220 through the vasculature. In some embodiments, the control unit 260 can be configured to receive data from the sensor(s) 228, e.g., pressure data or other physiological data. In some embodiments, the control unit 260 can be configured to communicate the data to a remote compute device, e.g., via a wired or wireless connection. In some embodiments, the control unit 260 can be configured to analyze the data, as further described with reference to FIG. 18. Additional details of a control unit are provided with reference to FIG. 16. In some embodiments, the control unit 260 can be configured to control catheter torquability via a user interface (e.g., deployment system, control unit, etc.). In some embodiments, the control unit 260 may include the control unit 2500 described with respect to FIG. 25.

The system 200 includes an optional receptacle 280 configured to receive and/or secure the housing. In some embodiments, the receptacle 280 can be configured to provide power to one or more components in the system 200, such as the guide deployment mechanism 236, the advancement mechanism 238, the control unit 260, the braking mechanism 290, or the retraction mechanism 292. In some embodiments, the housing 210 can be mechanically coupled to the receptacle 280 via, e.g., mating components (e.g., slot or opening that mate with a corresponding protrusion or other structure). In some embodiments, the housing 210 can be magnetically coupled to the receptacle 280 via, e.g., permanent magnet(s) and/or solenoid(s).

One or more components of the system 200 (e.g., control unit 260, advancement mechanism 238, guide deployment mechanism 236, braking mechanism 290, retraction mechanism 292) can be integrated into and/or coupled to (e.g., attached to) the receptacle 280 and/or housing 210. In some embodiments, the deployment mechanism 236, the advancement mechanism 238, and/or the optional components 260, 290, and 292 are disposed outside the housing 210 as illustrated in FIG. 6. In some embodiments, the deployment mechanism 236, the advancement mechanism 238, and/or the optional components 260, 290, and 292 can be disposed within the housing 210 and outside the chamber 212. In these embodiments, the housing 210 substantially encloses these components and the resulting system 200 can be more compact.

In some embodiments, the advancement mechanism 238, and/or the optional components 260, 290, and 292 can be separate from the housing 210. For example, the housing 210 can be received by a first portion of the receptacle 280 and other components (the advancement mechanism 238, and/or the optional components 260, 290, and 292) can be received by a second portion of the receptacle 280. One or more wires or cables can be disposed within the receptacle 280 to operatively couple the housing 210 (and the catheter 220) with the other components.

FIG. 7 shows a schematic of an example system 300 for RHC, showing a coupling between a connector 318 of the system 300 and a hub 352 of an IV catheter, according to embodiments. The system 300 can include components that are structurally and/or functionally similar to those of system 200 described above. For example, the system 300 includes a housing 310 and a catheter 320 that can be coupled to an IV catheter 350 via a connector 318. This coupling is schematically depicted using arrow B in FIG. 7. The IV catheter 350 can include a hub 352 and a needle 354 that ends in a distal tip 356 which is positionable in a vessel V. When coupled, the catheter 320 can be advanced through a lumen of the IV catheter 350 and into the vessel V, such that the catheter 320 can be further navigated through the vasculature to the right side of the heart. As depicted, the housing 310 contains the catheter 320 within a limited sterile field. In operation, remote computer systems and other connections to the catheter 320 (e.g., via one or more ports 314 on the housing 310) can be outside of the sterile field, while the catheter 320 is contained within the housing 310 in a sterile area and a sterile coupling can be established between the connector 318 and the IV catheter 350.

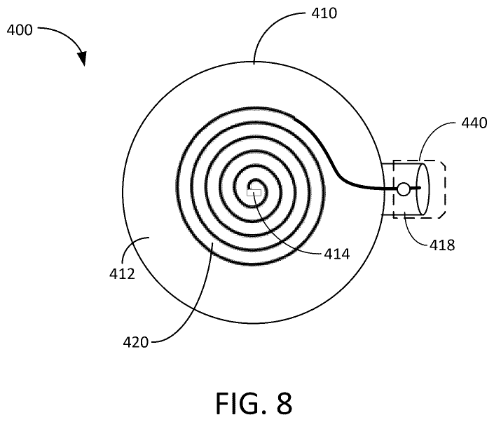

As described above, a catheter (e.g., catheter 120, 220, 320) can be stored within a housing (e.g., housing 210, 310) according to a variety of arrangements. For example, FIG. 8 shows a schematic of a system 400 including a catheter 420 stored in a spooled arrangement, according to an embodiment. The system 400 can include components that are structurally and/or functionally similar to other systems described herein (e.g., systems 200, 300). The system 400 includes a housing 410 that is configured to enclose a catheter 420 wound or coiled about a spool. In some embodiments, the resulting catheter coil has a diameter of about 20 cm or less (e.g., about 20 cm, about 18 cm, about 16 cm, about 14 cm, about 12 cm, about 10 cm, or less, including any values and subranges in between). The system 400 may include a sterile casing 412 that is configured to establish a sterile field for the catheter in the housing 410.

Similar to other housings described herein, the housing 410 can include a connector 418 for connecting to an IV catheter and through which the catheter 420 can be advanced out of the housing 410. The system 400 can also include one or more additional ports or connectors 414 for enabling other connections to a proximal end of the catheter 420. In some embodiments, the ports or connectors 414 can be connected to one or more lumens, shafts, etc., defined by the catheter 420. In some embodiments, the ports or connectors 414 can be configured to receive a fluid and communicate the fluid into the catheter, e.g., for delivery into the vasculature and/or deployment of a guide of the catheter 420. In some embodiments, the ports or connectors 414 can be coupled to a wire or cable that is connected to one or more sensors disposed on the distal end of the catheter 420.

When not in use (e.g., during transport or storage), the connector 418 of the housing can be covered, e.g., via a cover (e.g., membrane or flap) or cap 440, to maintain the sterility of the connector 418 and the interior of the housing 410 prior to coupling to an IV catheter. In some embodiments, a cover or cap can also be used for the ports or connectors 414 so as to maintain the sterility of the environment surrounding the catheter 420.

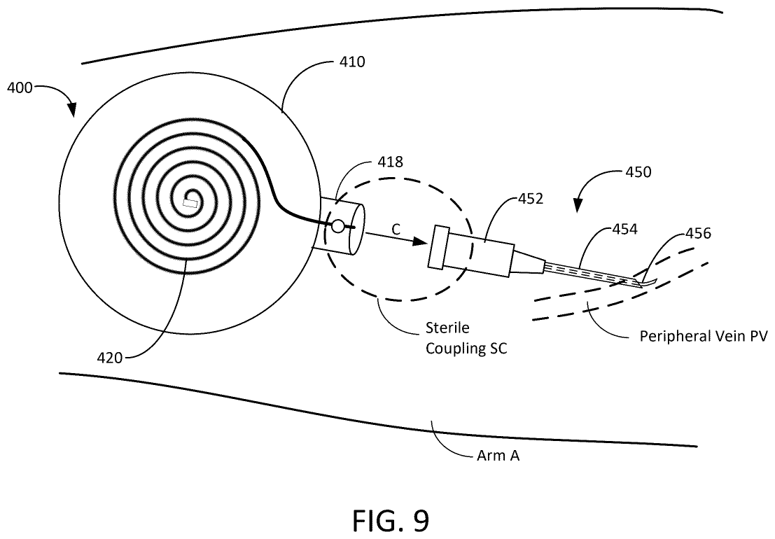

FIG. 9 depicts the system 400 being coupled to an IV catheter 450, according to an embodiment. The IV catheter 450 comprises a hub 452 and a needle 454, whereby the needle 454 extends into a peripheral vein PV (e.g., an antecubital vein) of an arm A. Once coupled, the catheter 420, which includes a distal tip 456, can be advanced through the IV catheter 450 and into the peripheral vein PV. A sterile coupling SC can be established between the connector 418 of the housing 410 and the hub 452 of the IV catheter 450, thereby ensuring sterility of the catheter 420 as it is advanced out from the housing 410 and into the peripheral vein PV. In some embodiments, the IV catheter 450 is an 15-gauge IV catheter or smaller catheter. In some embodiments, the IV catheter 450 can range from about 25-gauge to about 10-gauge, including all values and sub-ranges therebetween. While depicted as being disposed within a peripheral vein PV of an arm A in FIG. 9, it is appreciated that the IV catheter and therefore the system 400 (and other systems described herein) can be used to deliver a catheter into another vein of a subject, such as, for example, the internal jugular vein or the femoral vein, to perform the catheterization. As depicted in FIG. 9, the system 400 can be compact and portable (e.g., the dimension of the housing 410 can be about or less than a width of the arm of the patient), and the small dimension of the catheter 420 allows catheterization via a peripheral vein of the arm A. Accordingly, the catheterization can be readily conducted in an outpatient setting. For example, the catheterization can be conducted in an in-office (e.g., in-clinic) setting without anesthesia using a standard gauge IV (e.g., an 18 gauge IV or smaller).

Catheter devices as described herein can include one or more different types of guides. For example, FIGS. 10A and 10B depict undeployed and deployed states, respectively, of a guide 526 implemented as an expandable member (e.g., an inflatable balloon or self-expanding gel). The guide 526 can be part of a catheter 520, which can include components that are structurally and/or functionally similar to other catheters described herein. For example, the catheter 520 includes a body 522 that defines a lumen 537. FIG. 10A illustrates the guide 526 in an undeployed or resting state, e.g., for when the catheter 520 is stored and/or being inserted into the vascular system of the patient. The guide 526 can transition into a deployed state, where a volume or size of the guide 526 increases, as depicted in FIG. 10B. In the deployed state, the guide 526 can be used to catch the blood flow within a subject's vein, which can drive the advancement of the catheter 520 to the heart.

In some embodiments, the guide 526 can be a balloon, which can be transitioned into the deployed state, e.g., by delivering a fluid (e.g., liquid or gas) into the balloon via the lumen 537. In some embodiments, the guide 526 can be formed from a self-expanding substance, which can be delivered (e.g., via the lumen 537), released, or activated near a distal end of the catheter 520 to expand into its deployed state. For example, the guide 526 can include a self-expanding gel or foam, which can transition into the deployed state via contact with water or another appropriate reactant (e.g., such as one delivered through the lumen 537). In some embodiments, the gel or foam can dissolve after the catheterization procedure, e.g., to facilitate retraction of the catheter. In some embodiments, the guide 526 made of the gel or foam can be dissolved by applying an electrical current to the guide 526. In some embodiments, the guide 526 made of the gel can be dissolved by applying a light beam or pulse to the gel. The light beam or pulse can be transmitted to the guide 526 via, for example, an optical fiber disposed within the catheter body 522. In some embodiments, the guide 526 made of the gel can be dissolved due to an increase in temperature.

FIGS. 11A and 11B depict undeployed and deployed states, respectively, of a guide 626 implemented as a sail, according to an embodiment. The guide 626 can be part of a catheter 620, which can include components that are structurally and/or functionally similar to other catheters described herein. For example, the catheter 620 can include a catheter body 622 and a shaft 639 extending through the catheter body. As depicted in FIG. 11A, in the undeployed state, the surface of the guide 626 remains close (e.g., substantially parallel) to the catheter body 622, e.g., to reduce a lateral profile of the guide 626. In some embodiments, the shaft 639 can slide along the length of the catheter body 622 to unfold the sail, as depicted in FIG. 11B. In the deployed state, the surface of the guide can form a dome 626a (also referred to as a canopy) such that the blood flow of the patient can be caught by the sail and drive the sail toward the heart. Once at the heart, the guide 626 can also facilitate navigation of the catheter 620 through the heart chambers and into and/or through the pulmonary arteries. The guide 626 can be formed from any suitable viscoelastic material or a selectively expandable material, for example, a hydrogel, a polymer (e.g., a block co-polymer), a solgel, a silicone gel, any other suitable material or a combination thereof. As depicted in FIG. 11A and 11B, the direction of blood flow of in the vessel of a patient within which the guide 626 is deployed is from left to right. In some embodiments, the direction of blood flow may be from right to left. It can be appreciated that while guide 626 depicts a particular design, any of the designs of the guides shown herein can be combined together in a guide assembly.

FIGS. 12A and 12B depict undeployed and deployed states, respectively, of a guide 1526 implemented as a parachute, according to an embodiment. The guide 1526 is disposed on a catheter 1520 that includes a catheter body 1522 and a plurality of suspension lines 1539. In the undeployed state as depicted in FIG. 12A, the suspension lines 1539 can be held close to the catheter body 1522 and the surface of the parachute can be folded. In the deployed state as depicted in FIG. 12B, the suspension lines 1539 can be released and blood flow can catch the parachute to unfold the parachute into a canopy 1526a. Accordingly, the blood flow of the patient can drive the advancement of the deployed parachute. The guide 1526 can be formed from any suitable viscoelastic material or a selectively expandable material, for example, a hydrogel, a polymer (e.g., a block co-polymer), a solgel, a silicone gel, any other suitable material or a combination thereof. As depicted in FIG. 12A-12B, the direction of blood flow in the vessel of a patient within which the guide 1526 is deployed is from left to right. In some embodiments, the direction of blood flow may be from right to left. It should be appreciated that while guide 1526 depicts a particular design, any of the designs of the guides shown herein can be combined together in a guide assembly.

In some embodiments, the guides 526, 626, and 1526 can be transitioned between the undeployed and deployed states (e.g., transitioned from the undeployed state into the deployed state and back into the undeployed state), e.g., by removing an inflation fluid, sliding back a shaft or line (e.g., the shaft 639 or suspension lines 1539), etc. In some embodiments, the catheter can include a return mechanism (e.g., a spring or actuator), that can be actuated to transition the guide back to the undeployed state.

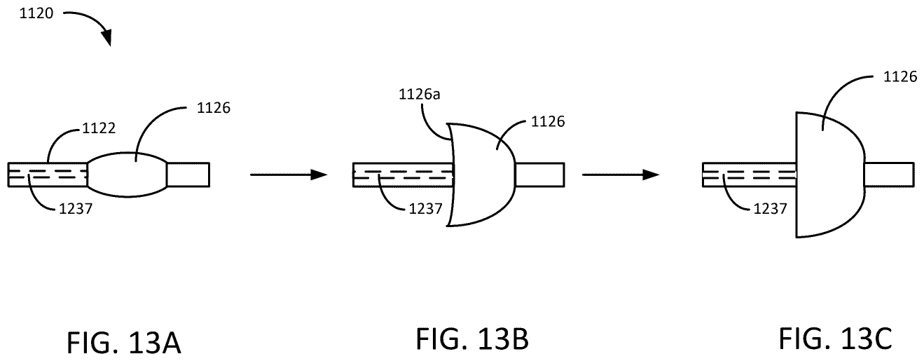

In some embodiments, a guide can be transitioned between multiple configurations, including those intermediate to a fully closed and a fully deployed configuration. For example, FIGS. 13A to 13C depict a guide 1126 implemented as a sail that can transition between three configurations, according to an embodiment. The guide 1126 is disposed on a catheter 1120 that includes a catheter body 1122. A shaft 1237 is disposed within the catheter 1122 to control the operation of the guide 1126, including, e.g., transitioning the guide 1126 between different configurations. For example, the guide 1126 can be in a first configuration when being advanced into the vasculature, as depicted in FIG. 13A. In this first configuration, the guide 1126 is in the undeployed state. The guide 1126 can be in a second configuration when being used to navigate to a target vessel in the heart, as depicted in FIG. 13B. In this second configuration, the guide 1126 is in a deployed state where a portion of the guide 1126 can form a cavity 1126a or dome shape that can catch the blood flow and be carried by the blood flow through the vasculature of the patient.

FIG. 13C depicts the guide 1126 in a third configuration. The guide 1126 can be transitioned into the third configuration, for example, to occlude a vessel or anchor a distal end of the catheter 1120. For example, the guide in the third configuration can be used to occlude a branch of a pulmonary artery, e.g., for collecting PCWP measurements. The guide 1126 in the third configuration can have a diameter that is greater than the guide 1126 in the first and second configurations. The diameter of the guide 1126 in the third configuration can be sized to occlude a vessel (e.g., branch of pulmonary artery) while the diameter of the guide 1126 in the first and second configurations can be sized to travel alone one or more vessels. The guide 1126 can be formed from any suitable viscoelastic material or a selectively expandable material, as previously described. As depicted in FIG. 13A to 13C, the direction of blood flow in the vessel of a patient within which the guide 1126 is deployed is from left to right. In some embodiments, the direction of blood flow may be from right to left. It should be appreciated that while guide 1126 depicts a particular design, any of the designs of the guides shown herein can be combined together in a guide assembly.

In some embodiments, a guide (e.g., the guide 1126 or other guides described herein) can be transitioned between more than three configurations (e.g., four configuration, five configurations, or more). In some embodiments, the guide can be transitioned in a continuous manner between the deployed state and the undeployed state. Such transitioning can be used, for example, to control a speed of movement of the guide through the vasculature.

FIGS. 14A to 14C depict a guide 726 that can be configured as a sample collection element, according to an embodiment. The guide 726 is disposed on a catheter 720 that includes a catheter body 722. FIG. 14A depicts that the guide 726 is in a first configuration (e.g., undeployed state) and FIG. 14B depicts that the guide 726 is in a second configuration (e.g., deployed state). In the second configuration, the surface of the guide 726 forms a canopy 726a such that the blood flow of the patient can drive the motion of the catheter 720 through the vasculature. FIG. 14C depicts that the guide 726 is in a third configuration (also referred to as a sample collection state). In this third configuration, the guide 726 can form a container 726b to collect and hold a sample (e.g., blood sample). In some embodiments, the guide 726 can form the container 726b by flipping or reverting (e.g., folding over) the canopy 726a in the second configuration. The guide 726 can be formed from any suitable viscoelastic material or selectively expandable material, as previously described. As depicted in FIG. 14A to 14C, the direction of blood flow in the vessel of a patient within which the guide 726 is deployed is from left to right. In some embodiments, the direction of blood flow may be from right to left. It should be appreciated that while guide 726 depicts a particular design, any of the designs of the guides shown herein can be combined together in a guide assembly.

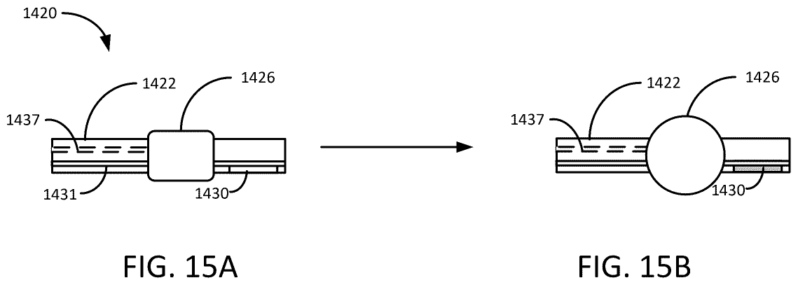

FIGS. 15A and 15B depict the operation of a guide 1426 and a sample collection element 1430, according to an embodiment. The guide 1426 and the sample collection element 1430 are disposed on a catheter 1420 that includes a catheter body 1422. The catheter body 1422 defines a first lumen 1431 connected to the sample collection element 1430 and a second lumen 1437 connected to the guide 1426. FIG. 15A shows that the guide 1426 is in the undeployed state and the sample collection element 1430 is empty. FIG. 15B shows that the guide 1426 is transitioned to the deployed state. Once the guide 1426 navigates the catheter tip to a target location (e.g., pulmonary blood vessel), the sample collection element 1430 is configured to collect and/or hold a sample (e.g., blood). The guide 1426 can be formed from any suitable viscoelastic material or selectively expandable material, as previously described herein. As depicted in FIGS. 15A and 15B, the direction of blood flow in the vessel of a patient within which the guide 1426 is deployed is from left to right. In some embodiments, the direction of blood flow may be from right to left. It should be appreciated that while guide 1426 depicts a particular design, any of the designs of the guides shown herein can be combined together in a guide assembly.

FIG. 16 shows a schematic of a system 1300 associated with the catheters described herein, according to an embodiment. The system 1300 can include one or more components that are structurally and/or functionally similar to other systems described herein (e.g., systems 200, 300). For example, the system 1300 can include a control unit 1360. The control unit 1360 can be coupled to sensor(s) 1328, which can be disposed on the distal end of a catheter. In some embodiments, the system 1300 can also include sensors(s) 1328 that are disposed on or near a proximal end of the catheter, such as, for example, a sensor to read one or more markers on the catheter to monitor the navigation progress of the catheter. The communication between the control unit 1360 and the sensors(s) 1328 can be bidirectional, e.g., the control unit 1360 is configured to receive and/or analyze data acquired by the sensor(s) 1328 and send signals to the sensor(s) 1328 (e.g., control signals).

The system can include a user interface 1370, which can be operatively coupled to the control unit 1360. The user interface 1370 is configured to allow communication between a user and the control unit 1360. In some embodiments, the user interface 1370 includes an optional display 1372, e.g., configured to display data or analysis results sent from the control unit 1360. For example, the display 1372 can display the pressure wave forms acquired by pressure sensors (e.g., sensor(s) 1328) on a moving time scale. As described above, the pressure waves can be characteristic for each component of the vascular system being traversed by a catheter. Therefore, a user can determine a location of the catheter tip by viewing the pressure wave forms. In some embodiments, the display 1372 is configured to visually display the advancement of the catheter within the vascular system. For example, the display 1372 can be configured to display an anatomical map of a human (or part of the human such as the heart portion) and indicate the location of the catheter tip on the map, e.g., based on pressure data collected by sensor(s) 1328 disposed on the distal end of the catheter.

In some embodiments, the user interface 1370 includes an audio device 1374, e.g., configured to send out audio signals. The audio signals can include, for example, alert signals indicating one or more safety issues, recitations or other audio indications of data or results analyzed by the control unit 1360 (e.g., progress of the catheter within the vascular system of the patient), indicators that inform the user of the start/stop of the catheterization process and/or location of the catheter as it advances through the vasculature, and/or instructions to the user when/how to proceed throughout the procedure based on the analysis of the position of the catheter tip.

In some embodiments, the user interface 1370 includes an optional input mechanism (e.g., keyboard, mouse, touch screen, etc.) such that a user can enter information into the system 1300. For example, the user can enter instructions into the system 1300 to start/stop deploying the catheter, and such instructions can be based on the output of the user interface 1370 (e.g., as conveyed by the display 1372 and/or audio device 1374). In another example, the user can enter instructions into the system 1300 to control the operation of the sensor(s) 1328 (e.g., start/stop collecting data, change data acquisition rate, etc.). The inputs made by the user can be received by the control unit 1360, which can control the operation of one or more components of the system 1300 based on the inputs.

In some embodiments, the control unit 1360 can be optionally coupled to a guide deployment mechanism 1336, an advancement mechanism 1338, a braking mechanism 1390, and a retraction mechanism 1392 (collectively referred as optional components). Similar to the optional components depicted in FIG. 6, these mechanisms are configured to control the deployment of the guide and/or the catheter. In some embodiments, the control unit 1360 can be operatively coupled to these components to control their operation. In other embodiments, one or more of these components can be manually operated by a user, e.g., via a slider, button, thumbwheel, flange, etc.

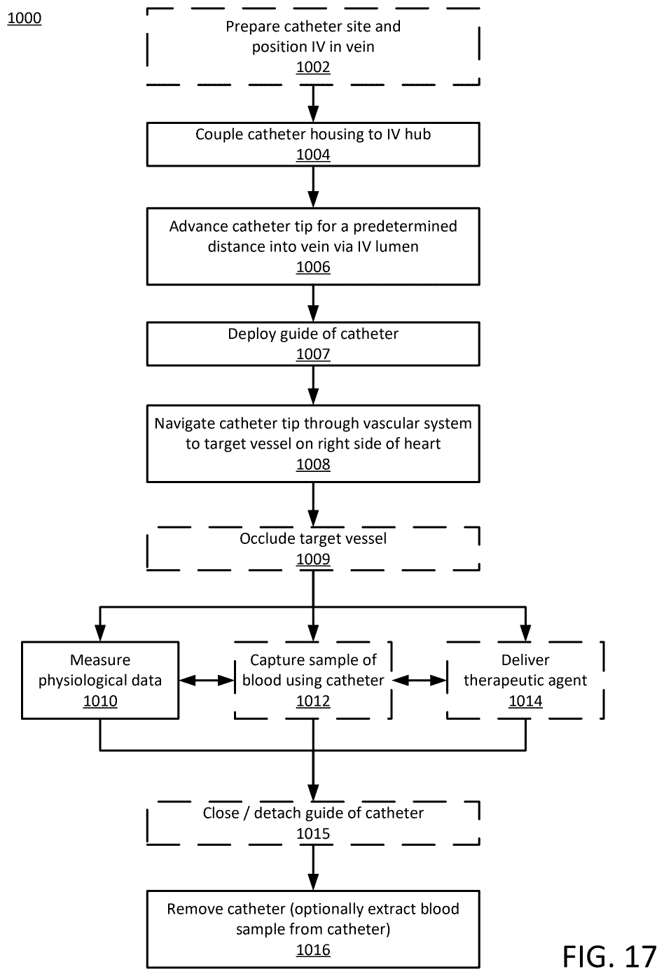

The control unit 1360 can include a processor 1362, a memory 1364, and an I/O interface 1308. In some embodiments, the memory 1364 is configured to store processor executable instructions (also referred to as codes), and upon execution of the processor executable instructions, the processor 1362 is configured to perform one or more methods described herein (e.g., methods described with reference to FIGS. 17 and 18). The memory 1362 includes a data collection unit 1365 configured to store processor executable instructions associated with data collection, and a data analytics unit 1366 configured to store processor executable instructions associated with data analytics. In some embodiments, the instructions in the data collection unit 1365 can be executed by the processor 1362 to control the operation of the sensors(s) 1328. In some embodiments, the instructions in the data analytics unit 1366 can be executed by the processor 1362 to analyze the data acquired by the sensor(s) 1328 and/or data stored in the memory 1364 (e.g., patient data, specification data of the catheter, etc.).

In some embodiments, the memory 1364 includes an optional device deployment unit 1367 configured to store processor executable instructions associated with device deployment. For example, the instructions in the device deployment unit 1367 can be executed by the processor 1362 to control the deployment of the catheter via, e.g., the advancement mechanism 1338, the braking mechanism 1390, and/or the retraction mechanism 1392. In some embodiments, the instructions in the device deployment unit 1367 can be executed by the processor 1362 to control the deployment of a guide in the catheter via, e.g., the guide deployment mechanism 1336.

In some embodiments, the control unit 1360 can also be operatively coupled to one or more optional compute device(s) 1378 (e.g., that are remote from the catheter and housing, as described in embodiments herein). In some embodiments, the compute device(s) 1378 can be configured for data analysis, data storage, and/or other functionality. For example, the compute device(s) 1378 can be configured to receive data collected by sensor(s) 1328 from the control unit 1360 and to store that data and/or perform data analysis of that data.

In some embodiments, the compute devices(s) 1378 include one or more servers that are coupled to the control unit 1360 via a network connection (e.g., wired or wireless network). In some embodiments, the compute device(s) 1378 can be coupled to multiple control units similar to the control unit 1360 and configured to analyze data sent by these control units (e.g., big data analysis). For example, the compute device(s) 1378 can collect data about multiple patients and/or multiple catheterization procedures, e.g., to analyze the therapeutic effect of a medicine.

FIG. 17 is a flowchart illustrating an example method 1000 of operating catheters described herein, according to an embodiment. The method 1000 optionally includes, at 1002, preparing a catheter site and positioning an IV catheter in a vein. In some embodiments, the catheter site can be located on an arm of a patient. In some embodiments, the catheter site can be another appropriate site described with reference to FIG. 3A. The IV catheter includes a hub and an IV lumen. In some embodiments, the IV is an 18-gauge catheter or smaller catheter.

In some embodiments, preparing the catheter site includes sterilizing the catheter site and the area around the catheter site, and then inserting the IV catheter into the vein at the catheter site. In some embodiments, an IV catheter or other port providing access to a vein may already be present, e.g., due to a separate medical procedure, and therefore a catheter site does not need to be prepared.

At 1004, the housing of the catheter is coupled to the IV hub via a sterile coupling. The housing is configured to store the body of the catheter in a sterile field or environment. Coupling the housing of the catheter to the IV hub allows deployment of the catheter body out of the housing and through the IV lumen that is connected to the IV hub, while maintaining sterility of the catheter.

The method 1000 includes, at 1006, advancing the tip of the catheter for a predetermined distance into the vein via the IV lumen. In some embodiments, the distance of the catheter tip into the vein can be determined by reading the marker(s) on the catheter body (e.g., markers 143 and/or 144 as described with reference to FIG. 2). In some embodiments, the distance of the tip into the vein can be determined by the user. In some embodiments, the distance of the tip into the vein can be determined by a processor (e.g., the processor 1362 and/or the compute device(s) 1378).

Once the predetermined distance or a predetermined location is reached, the guide of the catheter is deployed, at 1007. In some embodiments, the guide can be deployed by delivering a fluid into the guide that is implemented as a balloon. In some embodiments, the guide can be deployed by unfolding the guide that is implemented as a sail or a parachute. Other guides as described herein can be deployed using different electrical, mechanical, etc. mechanisms.

At 1008, the catheter is navigated through the vascular system of the patient to arrive at a target vessel via the right side of the heart. In some embodiments, the target vessel includes a pulmonary artery branch. The movement of the catheter can be driven by blood flow of the patient with a guide of the catheter in a deployed state. In some embodiments, the location of the catheter tip within the vascular system is determined by the pressure wave forms acquired by one or more pressure sensors disposed on the distal end of the catheter (see, e.g., descriptions with reference to FIG. 3B). In some embodiments, the location of the catheter tip within the vascular system can be determined based on a combination of the pressure wave forms and the reading of the marker(s) on the catheter body. For example, the reading of the marker(s) can provide a rough determination of the catheter tip location and the pressure wave form can refine the determination. In some embodiments, other physiological measurements can be made by one or more sensors of the catheter as the catheter is navigated through the vasculature. For example, gas concentrations, cardiac signals, temperature, etc. can be measured as the catheter advances through the vasculature.

In some embodiments, the pressure sensor disposed at the distal end of the catheter is configured to continuously or periodically send measured signals to the control unit (e.g., at a repetition rate of every half second or higher). In some embodiments, a user interface (e.g., the user interface 1370) is used to display the location of the catheter tip based on the pressure wave forms acquired by the pressure sensor and the user can proceed or pause the navigation of the catheter tip based on the displayed location of the catheter tip.

In some embodiments, the method 1000 includes an optional step 1009, where the target vessel is occluded, e.g., to facilitate subsequent measurements (e.g., a measurement of PCWP). In some embodiments, the target vessel is occluded by the guide. For example, the guide can increase its volume from the deployed state to occlude the target vessel (e.g., by delivery of more fluid into the balloon that is configured as the guide). In some embodiments, the target vessel can be occluded by a separate occlusion element disposed at the catheter tip.

Once at the target vessel, the catheter can be used to perform one or more actions, at 1010 to 1014. In some embodiments, the method 1000 includes, at 1010, measuring physiological data of the patient. For example, the pressure sensor can be configured to measure the pressure and the measured pressure is the representative of the right atrial pressure of the patient. In some embodiments, the catheter includes an oxygen sensor (or other gas sensor) disposed at the distal end of the catheter and configured to measure the oxygen saturation (or other gas concentration) in the target vessel. In some embodiments, the catheter includes a pH meter configured to measure the pH value of blood in the target vessel. In some embodiments, the catheter includes a temperature sensor configured to measure the temperature in the target vessel. In some embodiments, the catheter can include multiple types of sensors that can be used to measure different physiological data of the patient.

In some embodiments, the method 1000 includes an optional step 1012 to capture a blood sample from the target vessel, e.g., using a sample collection element (such as a guide or a separate sample collection reservoir or chamber, as depicted in FIGS. 14A to 14C and 15A and 15B). In some embodiments, the method 1000 includes an optional step 1014 to deliver a therapeutic agent to the target vessel. The agent can be delivered through a lumen and a distal port defined by the catheter body (see, e.g., FIG. 2).

In some embodiments, the method 1000 includes an optional step 1015 to close or detach the guide of the catheter. For example, the guide can be transitioned back into an undeployed state to remove the catheter, or the guide can be released (e.g., a biodegradable gel or foam) prior to retracting the catheter from the vasculature. At 1016, the catheter is retracted from the vascular system of the patient. In some embodiments, one or more blood samples are collected and the method 1000 includes extracting the collected blood sample(s). In some embodiments, the guide is transitioned into the undeployed state and retracted out of the vascular system with the rest of the catheter. In some embodiments, the guide is left within the vascular system and configured to dissolve (e.g., biodegrade) afterwards.

FIG. 18 is a flowchart illustrating an example method 1600 of data analytics associated with operating catheters described herein, according to an embodiment. In some embodiments, the method 1600 can be performed by one or more processors (e.g., the processor 1362 and/or the compute device(s) 1378 in the system 1300 described with reference to FIG. 16).

The method 1600 includes, at 1602, receiving sensor data. In some embodiments, the sensor data includes data sent from a pressure sensor disposed at the distal end of a catheter. In some embodiments, the sensor data includes data sent from an oxygen sensor, a pH meter, a temperature sensor, or any other sensor described herein.

In some embodiments, the method 1600 includes an optional step of receiving patient data, at 1604. In some embodiments, the patient data can include physiological condition of the patient, such as the blood pressure, blood oxygen saturation level, age, gender, medical history, and existing condition(s), among others. In some embodiments, the patient data can be stored in a memory (e.g., the memory 1364 in the system 1300). In some embodiments, the patient data can be received from the compute device(s) 1378. In some embodiments, the patient data can be received from a user interface (e.g., the user interface 1370 in the system 1300).

At 1606, the received sensor data and/or the received patient data is analyzed. Based on the analysis, several possible actions can be taken. In some embodiments, the analysis at 1606 is configured to identify possible safety issues, at 1610. For example, the sensor data can be measured with the patient data to determine whether there is any aberration or deviation. In the event that a safety issue is identified, the catheter can be retracted (at 1612) and/or an alert/error message can be sent (at 1614). In some embodiments, the alert/error message can be sent to a server that is configured to store the patient data and the server is also accessible to other healthcare providers. In these embodiments, these health providers, such as the patient's primary care doctor or specialized doctor, can be informed of the condition of the patient.

In some embodiments, the data analysis at 1606 is configured to monitor the location of the catheter within the vascular system of the patient (at 1620). For example, the sensor data includes pressure measurements that can be used to indicate the location of the catheter tip (see, e.g., FIG. 3B). In the event that the catheter has not arrived at the target location (e.g., right heart), the method 1000 includes continuing the advancement of the catheter at 1622. On the other hand, in the event that the catheter has passed the target location, the method 1000 can include an optional step to retract the catheter (at 1624) to approach the catheter towards the target location. In some embodiments, the catheter has arrived at the target location and planned measurements have been completed (e.g., pressure measurement or sample collection), in which case the catheter can be retracted at 1624. The method 1000 also includes an optional step to output information regarding the catheter location at 1626. In some embodiments, the location information can be displayed on a user interface (see, e.g., FIG. 16).

In some embodiments, the data analysis at 1606 is configured to track patient information (at 1630). For example, the data analysis at 1606 can analyze the sensor data and/or the patient data to determine the physiological condition of the patient, including whether the condition of the patient improves or deteriorates, or whether the patient develops a new condition. The method 1000 also includes an optional step to send the patient information to one or more remote devices (at 1632). For example, the patient information can be sent to a server that is configured to store the medical record of the patient and/or the patient's healthcare provider(s).

Catheters described herein can have housing and advancement systems, as further described herein. Each of the systems depicted in FIGS. 19A to 26 can have components that are structurally and/or functionally similar to other systems described herein (e.g., systems 200, 300). FIGS. 19A and 19B depict two types of connection ports 1770 and 1870, respectively, that extend from the control unit and connect to the catheter housed 1710 within a sterile casing. FIG. 19A illustrates an intermittent junction design that allows for connecting and disconnecting the electricity from the catheter 1710 during the procedure. In some embodiments, the port 1770 allows for passage of four lumens and/or connections (e.g., air, injection fluid, electrical ports, etc.). While four lumens and/or connections are depicted in FIGS. 19A, it can be appreciated that any number of lumens, including less than four lumens or more than four lumens can be passed via port 1770. The top part 1771 shown in FIG. 19A is a fixed body attached to the deployment system (e.g., control unit, user interface) and controls the connection/disconnection as controlled by the user (e.g., physician). The bottom part 1711 shown in FIG. 19A is a rotating body that is connected to the rotation axis of the spool and allows for catheter 1710 advancement/retraction via a motor. In some embodiments, as depicted in FIG. 19B, the junction allows for a continuous connection without requiring disconnecting the fixed body from the rotating body. FIG. 19B illustrates a port system that utilizes a conductive brush that can contact the electrical lumens of the catheter and provide electrical connection (e.g., power to the sensors, receive data from sensors, etc.). The top body 1870 shown in FIG. 19B demonstrates the body that is connected to the control unit and is controlled by the user. The bottom body 1811 shown in FIG. 19B illustrates the part that is connected to the axis of rotation of the catheter 1810 and allows for catheter motion control (e.g., advancement/retraction, braking, speed, torquability, etc.).

Expanding further, FIG. 19A is a schematic illustration of a connection port 1770 that may be used to couple external leads, fluid, and/or gas ports to a catheter connector 1711. The catheter connector 1711 may be coupled to a proximal end of a catheter 1710 (e.g., any of the catheters described herein). The catheter connector 1711 includes a first channel 1712 formed about a central axis of the catheter connector, and a second channel 1714 that is radially offset from the central axis. In some embodiments, a first channel 1712 is a gas channel (e.g., an air channel), to allow communication of a gas to the catheter 1710, and the second channel 1714 may be a fluid channel, for example, for delivering medicaments through the catheter 1710 into the blood vessel, or drawing blood from the vessel. In some embodiment, the first channel 1712 may be the fluid channel, and the second channel 1714 may be the gas channel (e.g., air channel). The first channel 1712, as depicted, is centrally located and therefore can be configured to maintain constant coupling with port housing first channel 1772 (as described below). As such, the first channel 1712 can be selected to convey a gas, liquid, etc. where such connection is needed throughout the operation of the catheter. The catheter connector 1711 also includes a first electrical connector 1716 and a second electrical connector 1718 that are coupled to electrical leads (e.g., sensor leads) disposed through the catheter connector 1711.

The connection port 1770 includes a port housing 1771 defining (1) a port housing first channel 1772 configured to be coupled to the first channel 1712, and (2) a port housing second channel 1774 configured to be coupled to the second channel 1714. In some embodiments, the connection port 1770 may be coupled to the catheter connector 1711 via a Luer-lock, a snap-fit, or a friction-fit mechanism. The port housing first channel 1712 may be configured for gas communication and the port housing second channel 1714 may be configured for fluid communication, or vice versa. Alternatively, either one of the port housing first channel 1712 or the port housing second channel 1714 can be configured for gas communication or for fluid communication, including both channels being configured for gas communication or both channels being configured for fluid communication. The connection port 1770 also includes a port housing first electrical connector 1776 and a port housing second electrical connector 1776 configured to be electrically coupled to the first electrical connector 1716 and the second electrical connector 1718, respectively (e.g., when the port housing first channel 1772 is coupled to the first channel 1712). In some embodiments, the connectors 1716, 1718, 1776, and 1778 may include contact pins configured to provide electrical coupling via contact of the electrical connectors 1716, 1718 with the port housing electrical connectors 1776, 1778, respectively. In some embodiments, the second channel 1714 and the port housing electrical connectors 1776, 1778 may be configured to be coupled to the port housing second channel 1774 and electrical connectors 1716, 1718, respectively in a particular angular orientation of the connection port 1770 relative to the catheter connector 1711. A user may rotate the connection port 1770 to selectively couple or uncouple the electrical connectors 1716, 1718 from the port housing electrical connectors 1776, 1778 by rotating the connection port 1770 relative to the catheter connector 1711. In some embodiments, using the central first channel 1712 and the first port housing first channel 1772 may beneficially allow the first channel 1712 and the central first channel 1772 to remain fluidly coupled to each other to allow communication of the gas into the catheter 1710, while allowing selective uncoupling of the second channel 1714 from the port housing second channel 1774, and/or the electrical connectors 1716, 1718 from the port housing electrical connectors 1776, 1778.

FIG. 19B is a schematic illustration of a connection port 1870 that may be used to couple external leads, fluid, and/or gas ports to a catheter connector 1811, according to an embodiment. The catheter connector 1811 may be coupled to a proximal end of a catheter 1810 (e.g., structurally and/or functionally similar to any of the catheters described herein). The catheter connector 1811 includes a first channel 1812 formed about a central axis of the catheter connector 1811. The first channel 1812 may be a gas channel (e.g., an air channel), to allow communication of air to the catheter 1810, and/or a fluid channel, as described with respect to the connection port 1870. The catheter connector 1811 also includes an electrical connector 1816 that is coupled to an electrical lead (e.g., a sensor lead) disposed through the catheter connector 1811.

The connection port 1870 includes a port housing 1871 defining a port housing first channel 1872 configured to be coupled to the first channel 1812, for example, via a Luer-lock, a snap-fit, or a friction-fit mechanism, as previously described. The port housing first channel 1812 may be configured for gas communication and/or fluid communication. The connection port 1870 also includes a port housing electrical connector 1876 configured to be electrically coupled to the electrical port 1816, respectively (e.g., when the port housing first channel 1872 is coupled to the first channel 1812). In some embodiments, the port housing electrical connector 1876 includes a brush connector (e.g., a metal or carbon brush). Because the electrical connector 1816 forms a continuous path (e.g., a circular path) that the port housing electrical connector 1876 can follow, once the connection port 1870 is coupled to the catheter connector 1811, rotation of the connection port 1870 relative to the catheter connector 1811 can maintain coupling between the port housing electrical connector 1876 and the electrical connector 1816, e.g., as the port housing electrical connector 1876 slides on the electrical connector 1816. Thus, different from connection port 1770, the connection port 1870 may be rotated relative to the catheter connector 1811 while still allowing the port housing electrical connector 1876 to remain electrically coupled to the electrical connector 1816, thus providing operational flexibility and maneuverability. In some embodiments, it can be desirable to have continuous monitoring of different physiological conditions within a patient's body, e.g., as a catheter navigates to the target site, is at the target site, and/or is being retracted back from the target site. As such, having a continuous electrical connection can facilitate such monitoring.

FIG. 19C is a schematic illustration of a connection port 1970 that may be used to couple external leads, fluid, and/or gas ports to a catheter connector 1911, according to an embodiment. The catheter connector 1911 may be coupled to a proximal end of a catheter 1910 (e.g., structurally and/or functionally similar to any of the catheters described herein). The catheter connector 1911 includes a first channel 1912 formed about a central axis of the catheter connector, and a second channel 1914 that is radially offset from the central axis. In some embodiments, the first channel 1972 is a gas channel (e.g., an air channel), to allow communication of air to the catheter 1910, and the second channel 1914 may be a fluid channel, for example, for delivering medicaments through the catheter 1910 into the blood vessel, or drawing blood form the vessel. In some embodiment, the first channel 1912 may be the fluid channel, and the second channel 1914 may be the air channel. A proximal end of the second channel 1914 is divided into a second channel first portion 1915a and a second channel second portion 1915b that are radially offset from the first channel 1912. The portions 1915a/b may have a semi-circular or otherwise circular shape so as to be disposed circumferentially around a portion of the first channel 1912. While shown in FIG. 19C as being divided into two semicircular portions 1915a/b, the second channel 1914 may be divided into any number of portions disposed circumferentially or annularly around the first channel 1912 (e.g., 3, 4, 5, 6, or even more).

The catheter connector 1911 also includes a first electrical connector 1916 and a second electrical connector 1918 that are coupled to electrical leads (e.g., sensor leads) disposed through the catheter connector 1911. Each of the first and second electrical connectors 1916 and 1918 have a continuous path (e.g., a circular path) and are disposed circumferentially or annularly around the first channel 1912 radially outward of the second channel portions 1915a/b. The first electrical connector 1916 may be disposed radially outward of second electrical connector 1918.

The connection port 1970 includes a port housing 1971 defining (1) a port housing first channel 1972 configured to be coupled to the first channel 1912, and (2) a port housing second channel 1974 configured to be coupled to the second channel 1914. Similar to the second channel 1914, a distal the port housing second channel 1975 that faces the catheter connector 1911 is also divided into first and second portions 1975a and 1975b or, as referred to herein, port housing second channel first and second portions 1975a and 1975b. In some embodiments, the port housing second channel first and second portions 1975a and 1975b may be substantially similar in shape and size to the first and second potions 1915a, 1915b of the second channel 1914, as previously described. The semi-circular shape of the port housing second channel first and second portions 1975a, 1975b and the first and second portions 1915a, 1915b are configured to beneficially allow the connection port 1970 to be rotated relative catheter connector 1911, while still allowing the port housing second channel first and second portions 1975a and 1975b to overlap and remain fluidically coupled to at least one of the first portion 1915a and the second portion 1915b of the second channel 1914.

In some embodiments, the connection port 1970 may be coupled to the catheter connector 1911 via a Luer-lock, a snap-fit, or a friction-fit mechanism. The port housing first channel 1912 may be configured for gas communication and the port housing second channel 1914 may be configured to fluid communication, or vice versa, or both channels may be configured for gas communication, or both channels may be configured for fluid communication. The connection port 1970 also includes a port housing first electrical connector 1976 and a port housing second electrical connector 1976 configured to be electrically coupled to the first electrical connector 1916 and the second electrical connector 1918, respectively (e.g., when the port housing first channel 1972 is coupled to the first channel 1912). Similar to the port housing electrical connector 1876, the port housing electrical connectors 1976 and 1978 may include brush connectors, and are configured to remain in electrical contact with the electrical connectors 1916 and 1918, respectively, to maintain electrical communication therebetween in the instance that the connection port 1970 is rotated relative to the catheter connector 1911.

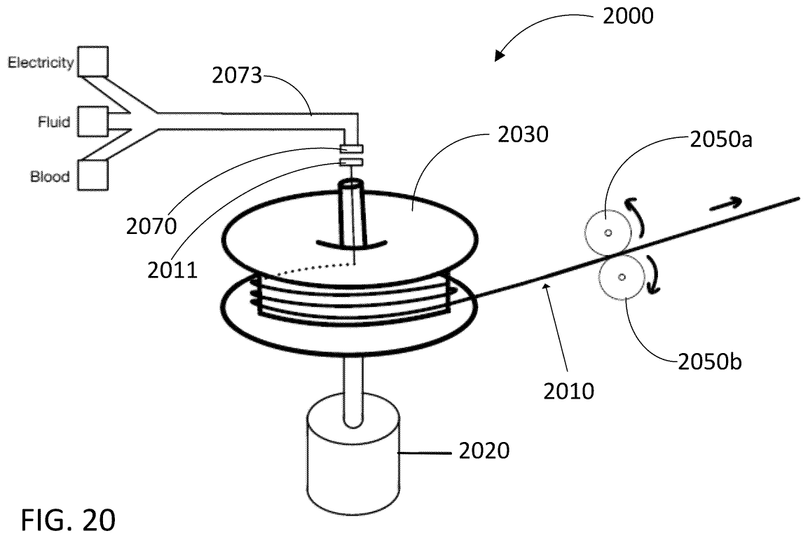

FIG. 20 depicts a housing implemented as a spool system 2000 for storing and deploying a catheter, according to an embodiment. In some embodiments, the system 2000 includes a base (not shown) to support an advancement/retraction mechanism implemented as a motor 2020 (e.g., a servo or a stepper motor) that is configured to rotate about an axis perpendicular to the surface of the base, as depicted in FIG. 20. The system 2000 also includes a spool 2030 and a catheter 2010 is wound on the spool 2030, as depicted in FIG. 20. The spool 2030 (together with the catheter 2010) can be placed onto the motor 2020. As the motor 2020 rotates along a first direction, the catheter 2010 on the spool 2030 is deployed out of the spool 2030. As the motor 2020 rotates along a second direction, opposite to the first direction, the catheter 2010 can be rewound onto the spool 2030 (i.e., retracting the catheter 2010). In some embodiments, the rotation of the motor 2020 can be calibrated such that the amount of catheter 2010 deployed out of the spool 2030 can be measured based on the number of rotations of the spool 2030. As shown in FIG. 20, a catheter connector 2011 may be coupled to an axial end of the catheter 2010, and a probe 2073 that couples the catheter 2010 to the control unit may include a connection port 2070 (e.g., structurally and/or functionally similar to the connection ports 1770, 1870, 1970, or any other connection ports described herein) configured to be coupled to the catheter connector 2011.

FIG. 20 also depicts a pair of rollers that gently grab the catheter 2010 as feeders 2050a and 2050b for storing and deploying a catheter 2010, according to an embodiment. As previously described, the catheter 2010 is wound about the spool 2030 for compact storage. The two feeders 2050a and 2050b are configured to rotate in opposite directions to draw the catheter 2010 out of the spool 2030 or retract the catheter 2010 that is already deployed. In some embodiments, the feeders 2050a/b may operate synchronously or asynchronously with the motor 2020 to facilitate feeding of the catheter 2010 at the desired rate. For example, the rotation of the spool 2030 can coordinate with the feeders 2050a and 2050b to deploy and/or retract the catheter 2010. In some embodiments, the feeders 2050a and 2050b can have the same configuration as the filament extrusion structure used in three-dimensional (3D) printing (e.g., additive printing). In some embodiments, the feeders 2050a and 2050b can be calibrated before use to ensure that a specified length of the catheter 2010 is deployed.

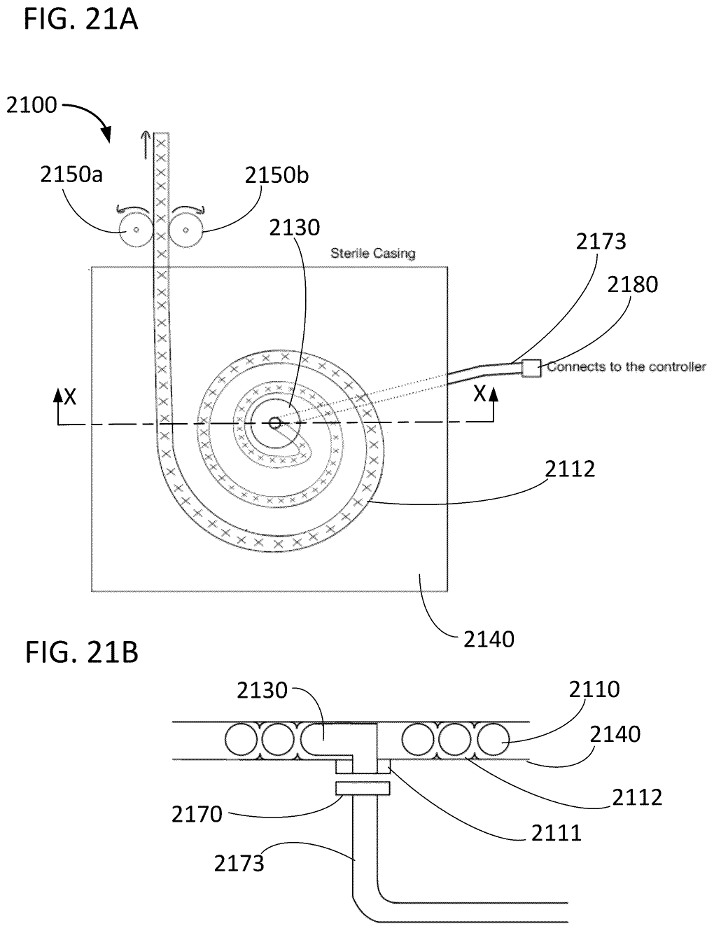

FIG. 21A depicts a system 2100 including a housing implemented as a sterile casing 2140 and two feeders 2150a and 2150b for storing and deploying a catheter 2110, according to an embodiment. The sterile casing 2140 includes a spiral channel 2112 configured to store the catheter 2110. The spiral channel 2112 can prevent the catheter 2110 from entanglement. In some embodiments, the spiral channel 2112 includes a sterile tube to maintain the sterility of the catheter 2110. The system 2100 also includes an axis of rotation in the center of the sterile catheter casing 2140 to allow for catheter 2110 advancement/retraction.

FIG. 21B depicts a cross-section side profile view of a portion of the spool system 2100 taken along the line X-X-in FIG. 21A, and shows the connection junction between the control unit (shown on top) and the catheter body (shown on bottom). As shown in FIG. 21B, the catheter connector 2111 may be coupled to an axial end of the catheter 2110, and a probe 2173 that couples the catheter 2110 to the control unit may include a connection port 2170 (e.g., the connection ports 1770, 1870, 1970, or any other connection ports described herein) configured to be coupled to the catheter connector 2111.

In some embodiments, a probe or other electrical connector 2130 is connected to the proximal end of the catheter 2110 and configured to extract data collected by the catheter 2110. The extracted data can be sent to a compute device, e.g., a processor, for further analysis, via the probe 2173. In some embodiments, the probe 2173 includes a wire or cable. In some embodiments, the probe 2173 is configured to follow the catheter 2110 as the catheter 2110 is deployed out of the sterile casing 2140. In some embodiments, the deployment of the catheter 2110 is conducted by the feeders 2150a and 2150b rotating in opposite directions.

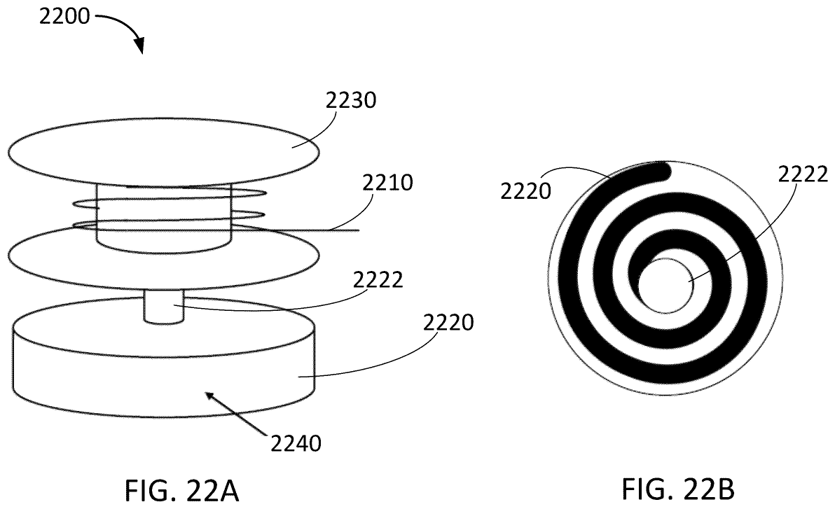

FIGS. 22A and 22B depict a system 2200 with a housing including a locking mechanism (e.g., braking mechanism) and a wound catheter, according to an embodiment. The system 2200 includes a base 2240, a shaft 2222, and a spool 2230. The shaft 2222 connects the base 2240 to the spool 2230 that holds a catheter 2210 wound as a coil. The proximal end of the catheter 2210 may include a connector (e.g., the catheter connector 1711, 1811, 1911, or any other catheter connectors described herein) configured to, e.g., for connection with a fluid source (e.g., to deploy a guide) and/or a signal source (e.g., to control the operation of one or more sensors). One or more springs (e.g., torsion springs) 2220 may be coupled to the spool 2230 via the shaft 2222. The system 2200 includes one or more actuators. For example, a first actuator (e.g., latch) may be configured to lock the catheter 2210 when a first button is pressed down from the control unit (e.g., deployment system), e.g., to stop further deployment of the catheter 2210. In some embodiments, releasing the first button on the deployment system (e.g., control unit, user interface, etc.) can retract the catheter 2210 under the force of the spring(s) 2220. In some embodiments, a second actuator may be configured to draw the catheter 2210 out of the coil when its corresponding button from the control unit (e.g., user interface, deployment system, etc.) is pressed down. Accordingly, the combination of the first actuator and the second actuator can be used to control the motion of the catheter (e.g., for deployment and retraction from the vasculature, torquing, etc.).

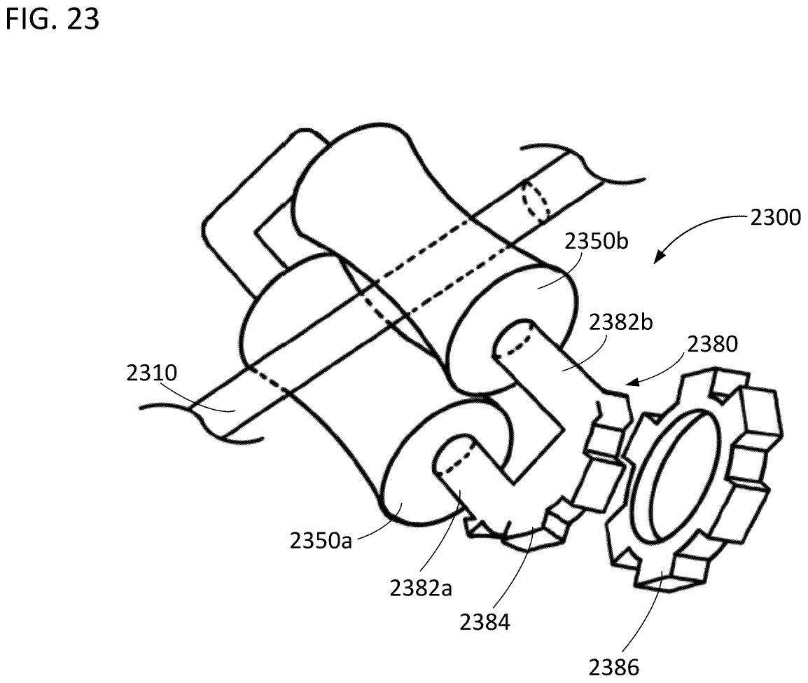

FIG. 23 depicts a system 2300 for controlling torque exerted on a catheter 2310, or any of the catheters described herein, according to an embodiment. The system 2300 includes a set of feeders 2350a and 2350b (e.g., rollers), each disposed along on an axis that is offset from each other, and that allow for controlling catheter 2310 torquability.

In some embodiments, the two feeders 2350a/b that gently grab the catheter 2310 are placed on an axis with a gear shaped side that is included in a torquing assembly 2380. For example, the torquing assembly 2380 may include a first axle 2382a on which the first feeder 2350a is mounted, and a second axle 2382b on which the second feeder 2350b is mounted. Corresponding axial ends of the axles 2382a and 2382b may be coupled together via a gear shaped member 2384. A torque gear 2386 may be coupled to or configured to be selectively coupled to the gear shaped member 2384, for example, by teeth of the gear shaped member 2384 meshing with corresponding teeth of the torque gear 2386. The torquing assembly 2380 is mounted within a housing (not shown) that permits rotation of the torquing assembly 2380 around the axis of the catheter 2310.

The torque gear 2386 can be controlled by the user (e.g., manually or via the control unit) to allow for catheter 2310 torquability. For example, rotation of the torque gear 2386 may cause the gear shaped member 2384 to rotate causing the feeders 2350a and 2350b to pivot around the axis of the catheter to thereby cause the catheter 2310 to rotate, thereby exerting a torque thereon. In some embodiments, the rotation can be within 0 degrees to 180 degrees of freedom, inclusive of all values and sub-ranges therebetween, to allow for clockwise or anticlockwise catheter rotation. As shown in FIG. 23, a surface of the feeders 2350a/b that contacts the catheter 2310 may be curved so as to provide a greater contact area between the feeders 2350a and 2350b and the catheter 2310. The contact between the feeders 2350a/b and the catheter 2310 can allow for advancing, withdrawing, bending, and/or rotating of the catheter 2310 by exerting a torque thereon. In some embodiments, the gear shaped member 2384 may include a circular member that extends circumferentially around the feeders 2350a/b. Such a gear-shaped member may allow rotation of the catheter 2310 up to an angle of 360 degrees or even higher, in either a clockwise or counterclockwise direction, thus allowing for greater torquability of the catheter 2310.

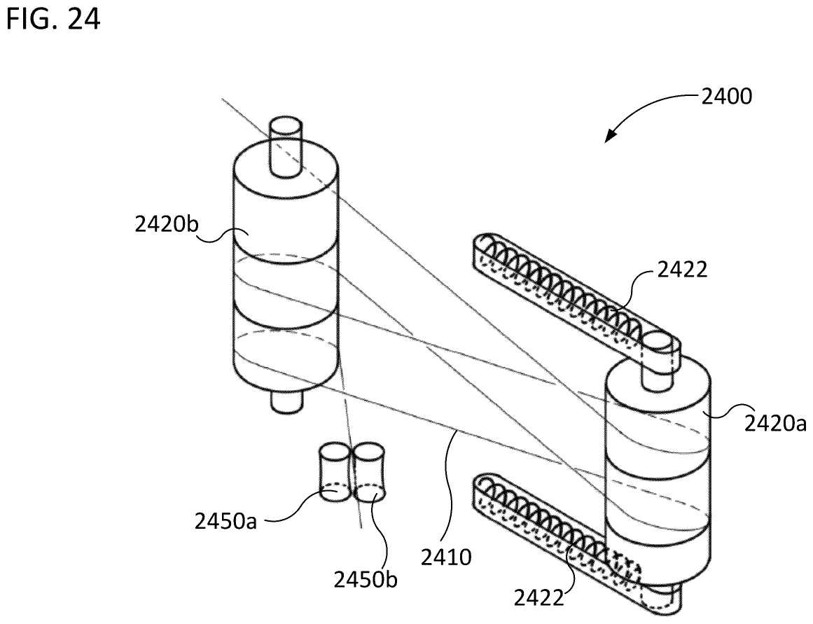

FIG. 24 depict a system 2400 with the catheter stored in an infinity loop arrangement, according to an embodiment. The system 2400 includes a first rotating pole 2420a (e.g., a rod or shaft) and a second rotating pole 2420b (e.g., a rod or shaft) as depicted by the isometric view in FIG. 24. A catheter 2410 is wound about these two poles 2420a and 2420b to form a coil having a bow (or infinity symbol) shape, when viewed from top, (also referred to as an infinity loop). In some embodiments, the two poles 2420a/b may be configured to selectively move closer to each other (e.g., via a linear rail movement mechanism 2422) as the catheter 2410 is advanced through the vasculature. The two poles 2420a/b can then become farther apart from each other during catheter 2410 retraction. The poles 2420a/b are segmented to allow for rotation of the segments at different speeds. In some embodiments, the catheter 2410 may include an adapter (not shown) at the proximal end thereof. The infinity loop of the catheter 2410 and the two poles 2420a and 2420b can be enclosed in a sterile housing, as previously described. Moreover, feeders 2450a and 2450b may be used to feed the catheter 2410 into a vasculature of a patient or withdraw the catheter 2410 therefrom. The movement of the feeders 2450a/b can be coordinated with the movement of the poles 2420a/b so as to feed or withdraw the catheter 2410 without inducing excessive strain or buckling of the catheter 2410.

FIG. 25 depicts a deployment system 2500 (e.g., control unit, user interface, data collection unit, etc.) that allows the user to control catheter deployment, advancement, and retraction (e.g., advancement and/or retraction, braking, speed, torquability, etc.), according to an embodiment. In some embodiments, the system 2500 includes four or less buttons to control catheter advancement/retraction and torquability. In some embodiments, the system 2500 includes a speed dial 2502 to control the catheter advancement/retraction speed during the procedure. In some embodiments, the deployment system 2500 may include a monitor 2504 that illustrates pressure waveforms (or other physiologic conditions including oxygen saturation level, pH, body temperature, etc.). In some embodiments, the deployment system may include catheter feed buttons 2506a and 2506b configured to control advancement or retraction of the catheter, respectively. In some embodiments, the deployment system 2500 may also include torquability buttons 2508a and 2508b, that may be configured to control rotation of the catheter (e.g., by actuating the system 2300 for controlling torque as depicted in FIG. 23) so as to control torque exerted on the catheter.

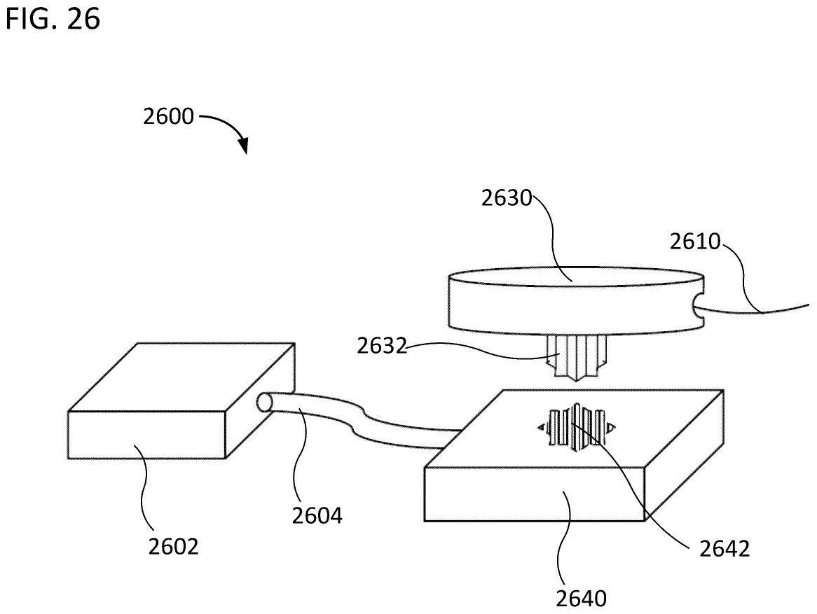

FIG. 26 depicts a system 2600 implemented as a cassette structure, according to an embodiment. The system 2600 includes a housing implemented as a spool 2630 and a catheter 2610 wound on the spool 2630. A receptacle implemented as a base 2640 can include one or more teeth 2642 (or other mating structure) for locking and/or securing the spool 2630 in place. The catheter 2610 can be deployed out of the spool 2630 by rotating the spool 2630. For example, a motor (not shown in FIG. 26) can be included in the base 2640 to drive the rotation of a geared shaft 2632 extending from spool 2630 into the receptacle 2640 to engage the one or more teeth 2642 (e.g., mesh therewith), which in turn rotates the spool 2630. In some embodiments, the base 2640 can contain a hollow core to allow for motor placement. In some embodiments, the motor can contain a hollow shaft. In some embodiments, a soft tubing 2604, containing lumens and electrical wires that mate to the catheter 2610 at an adapter (e.g., adapter 132, connection port 1970), can be used to protect a portion of the catheter 2610 and couple from the base 2640 to a deployment system 2602 (e.g., control unit, user interface, etc.). In some embodiments, the electrical and fluid lumens are routed through the hollow core of the motor so as to be connected to the catheter via the adapter (e.g., adapter 132, connection port 1970).

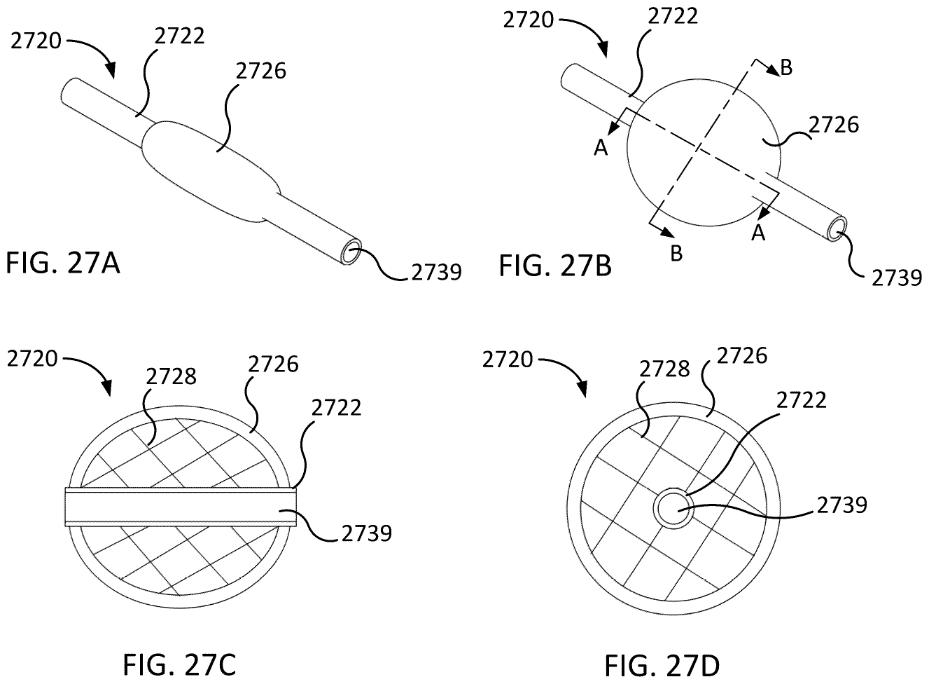

FIGS. 27A to 27D depict a catheter 2720 including a guide implemented as an expandable balloon 2726 filled with a self-expandable structure 2728 (e.g., self-expanding gel, viscoelastic material, hydrogel, silicone, etc.), according to an embodiment. FIG. 27A illustrates the undeployed state of the guide 2726. The guide 2726 can be coupled to or disposed on a catheter body 2722 that defines a lumen 2739. FIGS. 27B to 27D illustrate the deployed state of the guide 2726. The guide 2726 implemented as an expandable balloon can be filled with structures (e.g., self-expanding gel, viscoelastic material, hydrogels, a scaffold, or a shape memory alloy such as nitinol, or combination thereof). FIGS. 27C and 27D illustrate axial and radial cross-sections of the balloon 2726, respectively, in the deployed state, that are filled with the self-expandable structure 2728 to allow for an automatic expansion and collapse of the balloon 2726.

In some embodiments, the balloon 2726 is filled with a self-expandable structure 2728 (e.g., nitinol wires, linear polymer structures, etc.) to allow for an automatic deployment (e.g., expansion) and undeployment (e.g., contraction) as the catheter 2722 travels from smaller diameter veins (e.g., peripheral veins) to larger diameter veins (e.g., pulmonary artery), and vice versa. In some embodiments, the guide or balloon 2726 may be disposed in a sheath in an undeployed state, and is configured to move into a deployed state once the sheath is retracted to expose the guide 2726. In some embodiments, a biodegradable adhesive may be used to maintain the balloon 2726 in the undeployed state. The adhesive may be formulated to degrade with the vein of the user causing the balloon 2726 to move into the deployed state. Any other mechanism may be used to transition the balloon 2726 between the deployed and undeployed state, as previously described with respect to FIGS. 10A to 14C.

As depicted in FIGS. 27A, in the undeployed state, the balloon 2726 remains close to the catheter body 2722. The smooth surface of the balloon 2726 can allow for blood to flow around the balloon 2726 without stagnation. In some embodiments, the balloon 2726 may have a diameter in the deployed state in a range of about 4 mm to about 20 mm (e.g., 4, 5, 6, 7, 8, 9, 10, 11, 12, 13, 14, 15, 16, 17, 18, 19, or 20 mm, inclusive).

As depicted in FIGS. 27B to 27D, in the deployed state, the surface area of the guide 2726 can increase such that the blood flow (e.g., left to right or right to left in the FIGS. 27A to 27D) of the patient can be caught by the expandable balloon 2726 and drive the balloon 2726 toward the heart. Once at the heart, the balloon 2726 can also facilitate navigation of the catheter through the heart chambers and into and/or through the pulmonary arteries. In some embodiments, the self-expandable structure 2728 in the balloon 2726 enable the balloon 2726 to fold when automatically transitioning from the deployed state (FIGS. 27B to 27D) back to the undeployed state (FIG. 27A). Once the catheter 2722 reaches the desired location within a branch of the pulmonary artery, the balloon 2726 can automatically transition to the undeployed state (with a diameter of about 10 mm or less, e.g., about 10 mm, about 9 mm, about 8 mm, about 7 mm, about 6 mm, about 5 mm, about 4 mm, about 3 mm, about 2 mm, about 1.33 mm, about 1 mm, or less, including any values and subranges in between) or compress to a smaller size, e.g., to occlude a branch of the pulmonary artery for measuring the PCWP.

In some embodiments, the smooth balloon surface forms a closed profile that completely blocks blood flow through the balloon 2726, in both undeployed and deployed states, and allows for a better navigation to the heart by catching on the blood flow. The material of the expandable balloon 2726 allows for an automatic deployment and undeployment upon insertion of the catheter 2720 into a peripheral vein in the arm (e.g., antecubital fossa veins) through the vasculature up to the heart. The expandable balloon 2726 may not require any gas or fluid to be expanded. In some embodiments, the balloon 2726 may be porous or define a vent allowing fluid to enter or escape an inner volume of the balloon 2726, e.g., to activate the expandable structure 2728 and/or for balancing pressure during expansion and contraction of the balloon 2726. In some embodiments, the balloon 2726 may be formed of a material that degrades over time in blood to move the balloon 2726 into the undeployed state for retraction. It should be appreciated that while guide 2726 depicts a particular design, the guide 2726 can be combined with any of other designs described herein, or used in any of the systems described herein.

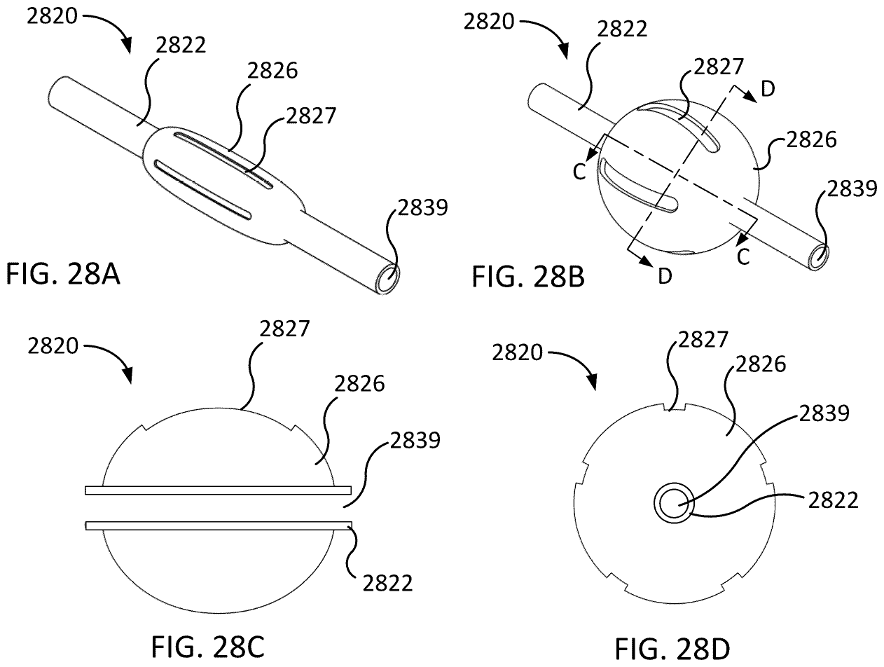

FIGS. 28A to 28D depict a catheter 2820 including a guide implemented as an expandable balloon 2826 with depressions or cutouts 2827, such as, for example, engraved slits, according to an embodiment. The catheter 2820 includes a catheter body 2822 defining a lumen 2839, and the balloon 2826 (e.g., formed of or including a self-expanding gel, viscoelastic material, hydrogel, silicone, etc.), including the slits 2827. In some embodiments, the balloon 2826 can a balloon that is not filled with structures, such as, for example, a solid balloon. Alternatively, in some embodiments, the balloon 2826 can include an inner structure similar to balloon 2726. FIGS. 28A and 28B illustrate the undeployed and deployed states, respectively, of the balloon 2826 in an isometric profile of the expandable balloon 2826 design with slits 2827. FIGS. 28C and 28D illustrate axial and radial cross-sections of the balloon 2826, respectively, in the deployed state.

The guide 2826 is disposed on the catheter body 2822 that defines a lumen 2839. As depicted in FIG. 28A, in the undeployed state, the balloon 2826 with slits 2827 remains close to the catheter body 2822. The slits 2827 can be more spread out or be positioned closer to each other on the surface of the balloon 2826 to allow for easier folding (e.g., collapse). Depending on the size, spacing, and/or geometric shape of the slides, the total number of the slits 2827 used on the surface of the balloon 2826 can vary. In some embodiments, the balloon 2826 may have a diameter in the deployed state in a range of about 4 mm to about 20 mm (e.g., 4, 5, 6, 7, 8, 9, 10, 11, 12, 13, 14, 15, 16, 17, 18, 19, or 20 mm, inclusive).

As depicted in FIGS. 28B to 28D, in the deployed state, the surface area of the guide 2826 can increase such that the blood flow (e.g., left to right or right to left in FIGS. 28A to 28D) of the patient can be caught by the expandable balloon 2826 and drive the balloon 2826 toward the heart. Once at the heart, the balloon 2826 can also facilitate navigation of the catheter through the heart chambers and into and/or through the pulmonary arteries. In some embodiments, the slits 2827 on the surface of the balloon 2826 allow for the blood to flow around the balloon 2826 body and enable the balloon 2826 to fold easier when automatically transitioning from the deployed state (FIGS. 28B to 28D) back to the undeployed state (FIG. 28A). Once the catheter body 2822 reaches the desired location within a branch of the pulmonary artery, the balloon 2826 can automatically transition to the undeployed state (with a diameter of about 10 mm or less, e.g., about 10 mm, about 9 mm, about 8 mm, about 7 mm, about 6 mm, about 5 mm, about 4 mm, about 3 mm, about 2 mm, about 1.33 mm, about 1 mm, or less, including any values and subranges in between) or compress to a smaller size, e.g., to occlude a branch of the pulmonary artery for measuring the PCWP. In some embodiments, the engraved slits 2827 on the balloon surface form a closed profile that completely blocks blood flow through the balloon 2827, in both undeployed and deployed states, and allows for a better navigation to the heart by catching on the blood flow. The material of the expandable balloon 2827 allows for an automatic deployment and undeployment upon insertion of the catheter 2820 into a peripheral vein in the arm (e.g., antecubital fossa veins) through the vasculature up to the heart.

In some embodiments, balloon 2826 may be in an undeployed state within a sheath, and moved into the deployed state by withdrawing the catheter 2820 out of the sheath. In some embodiments, the balloon 2826 may expand from undeployed state to the deployed state as the balloon 2826 moves from a smaller diameter to a larger diameter blood vessel such that the pressure exerted by walls of the smaller diameter vessel are sufficient to maintain the balloon 2826 in the undeployed state. In such embodiments, the balloon 2826 can have a strength or stiffness that is less than the blood vessel. In some embodiments, the balloon 2826 may be configured to move between the deployed and undeployed state due to hydration of the balloon 2826 as it absorbs the blood. In some embodiments, the balloon 2826 may be porous or define a vent allowing fluid to enter or escape an inner volume of the balloon 2826 for balancing pressure during expansion and contraction of the balloon 2826. In some embodiments, the balloon 2826 may be formed of a material that degrades over time in blood to move the balloon 2826 into undeployed state for retraction. It should be appreciated that while guide 2826 depicts a particular design, the guide 2826 can be combined with any of other designs described herein, or used in any of the systems described herein.

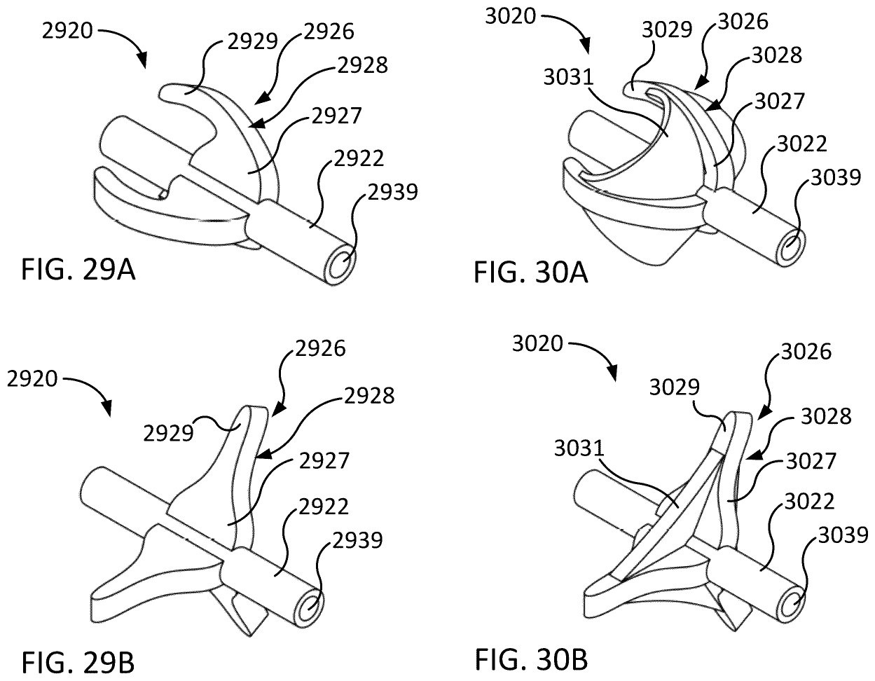

FIGS. 29A and 29B depict a catheter 2920 including a guide implemented as a self-expandable sail 2926 (e.g., self-expanding gel, viscoelastic material, hydrogel, silicone, etc.), according to an embodiment. FIGS. 29A and 29B illustrate the undeployed and deployed states, respectively, of the guide 2926 in an isometric profile of the expandable sail design. The guide 2926 is disposed on a catheter body 2922 that defines a lumen 2939. In some embodiments, the guide 2926 has petals 2928 that each include a first portion 2927 that is coupled to the catheter 2922, and a second portion 2929 extending radially away from the first portion 2927 in the expanded or deployed state of the petals 2928. The second portion 2929 may be thinner or have a smaller width than the first portion 2927 such that in the undeployed or contracted state, the second portion 2929 bends towards the catheter 2922, and in the deployed or expanded state, the second portion 2929 is substantially unbent and extends radially away from the first portion 2927. In some embodiments, the self-expandable sail 2926 may have a diameter in the deployed state of about 4 mm to about 20 mm (e.g., 4, 5, 6, 7, 8, 9, 10, 11, 12, 13, 14, 15, 16, 17, 18, 19, or 20 mm, inclusive).

The petals 2928 forming the sail 2926 may collapse upon insertion into the IV catheter (sheath) and automatically expand in the vasculature. As depicted in FIG. 29A, in the undeployed state, the petals 2928 of the sail 2926 remain close to the catheter 2922. The petals 2928 allow for easier undeployment (e.g., collapse) upon catheter 2922 insertion and a better deployment (e.g., expansion) to help navigate the catheter 2922 by catching on the blood flow. As depicted in FIG. 29B, in the deployed state, the surface area of the guide 2920 will increase such that the blood flow of the patient can be caught by the petals 2928 forming the expandable sail and drive the sail 2926 toward the heart. Once at the heart, the sail 2926 can also facilitate navigation of the catheter through the heart chambers and into and/or through the pulmonary arteries. Full collapse (e.g., folding) of the petals 2928 (FIG. 29A) can occlude a branch of the pulmonary artery for measuring the PCWP. The petals 2928 when fully collapsed can have a diameter of about 10 mm or less (e.g., about 10 mm, about 9 mm, about 8 mm, about 7 mm, about 6 mm, about 5 mm, about 4 mm, about 3 mm, about 2 mm, about 1.33 mm, about 1 mm, or less, including any values and subranges in between). The material of the expandable sail 2926 allows for an automatic deployment and undeployment upon insertion of the catheter 2922 into a peripheral vein in the arm (e.g., antecubital fossa veins) through the vasculature up to the heart. While FIG. 29A shows the second portion 2929 being bent towards the left direction (e.g., during insertion of the catheter 2920 into the vessel), in some embodiments, the second portion 2929 may bend in the opposite direction, i.e., towards the right (e.g., during withdrawal of the catheter 2920 from the blood vessel). In some embodiments, the sail 2926 may be formed of a material that degrades over time in blood to move the sail 2926 into the undeployed state for retraction. It should be appreciated that while guide 2920 depicts a particular design, the guide 2920 can be combined with any of other designs described herein, or used in any of the systems described herein.

FIGS. 30A and 30B depict a catheter 3020 including a guide implemented as an expandable sail 3026 with webbing 3031 (e.g., self-expanding gel, viscoelastic material, hydrogel, silicone, etc.), according to an embodiment. FIGS. 30A and 30B illustrate the undeployed and deployed states, respectively, of the guide 3026 in an isometric profile of the expandable sail design. The guide 3026 is disposed on a catheter body 3022 that defines a lumen 3039. The guide 3026 includes petals 3028 that may be substantially similar to the petals 2928, as previously described. In some embodiments, the guide 3026 has webbing 3031 in between the petals 3028 to allow for more surface area contact with the blood flow, for example, for better catching of the blood. As depicted in FIG. 30A, in the undeployed state, the petals 3028 of the sail 3026 and the webbing 3031 in between the petals 3028 remain close to the catheter body 3022. In some embodiments, the self-expandable sail 3026 with webbing 3031 may have a diameter in the deployed state of about 4 mm to about 20 mm (e.g., 4, 5, 6, 7, 8, 9, 10, 11, 12, 13, 14, 15, 16, 17, 18, 19, or 20 mm, inclusive).

As depicted in FIGS. 30B, in the deployed state, the webbing 3031 in between the petals 3028 allows for an increased surface area contact with the blood and helps navigate the catheter 3020 to the heart by catching on the blood flow. In some embodiments, the webbing 3031 serves two purposes, e.g., to allow for catching blood flow and/or help in wedging in a branch of the pulmonary artery once the catheter 3020 reaches the desired location in a branch of the pulmonary artery. In some embodiments, the webbing 3031 can provide a flat surface when the guide 3026 is in its undeployed state (FIG. 30A) in the pulmonary artery and occludes the blood flow for measuring the PCWP. The guide 3026 in the undeployed state can have a diameter of about 10 mm or less (e.g., about 10 mm, about 9 mm, about 8 mm, about 7 mm, about 6 mm, about 5 mm, about 4 mm, about 3 mm, about 2 mm, about 1.33 mm, about 1 mm, or less, including any values and subranges in between), or compress to a smaller size (e.g., when in the pulmonary artery).

In some embodiments, the webbing 3031 may be formed from the same material as the petals 3028. In some embodiments, the webbing 3031 may be formed from a different material from the petals 3028 (e.g., pig skin, artificially grown skin, etc.). While FIG. 30A shows the second portion 3029 being bent towards the left direction (e.g., during insertion of the catheter 3020 into the vessel), in some embodiments, the second portion 3029 may bend in the opposite direction, i.e., towards the right (e.g., during withdrawal of the catheter 3020 from the blood vessel). In some embodiments, the sail 3026 may be formed of a material that degrades over time in blood to move the sail 3026 into the undeployed state for retraction. It should be appreciated that while guide 3026 depicts a particular design, the guide 3026 can be combined with any of other designs described herein, or used in any of the systems described herein.

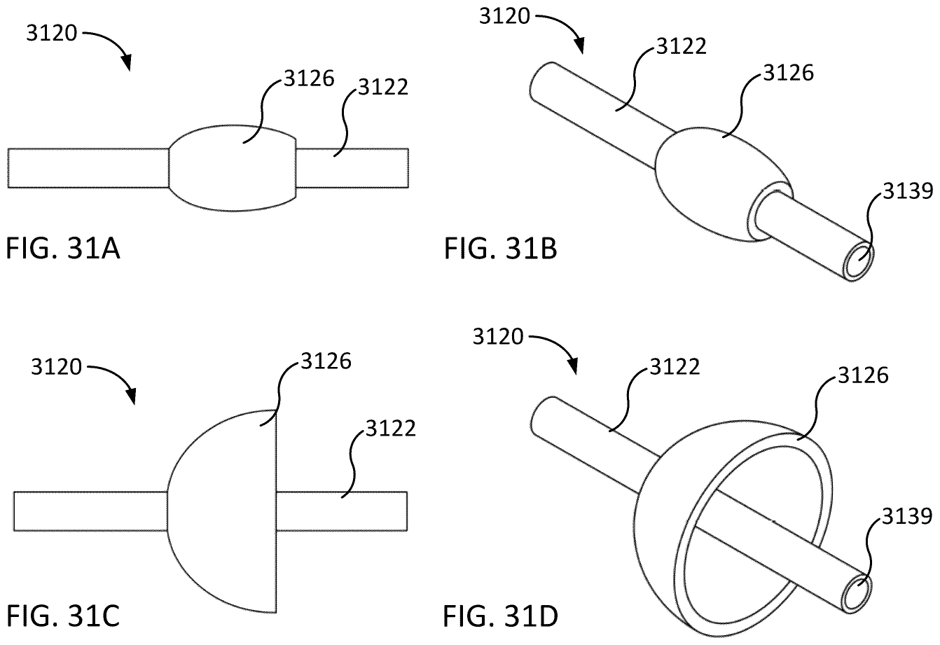

FIGS. 31A to 31D depict a catheter 3120 including a guide implemented as an expandable cup 3126 (e.g., self-expanding gel, viscoelastic material, hydrogel, silicone, etc.), according to an embodiment. FIGS. 31A and 31C illustrate the undeployed and deployed states, respectively, of the guide 3126 in a side profile. FIGS. 31B and 31D illustrate the undeployed and deployed states, respectively, of the guide 3126 in an isometric profile of the expandable cup design. The guide or cup 3126 is disposed on a catheter body 3122 that defines a lumen 3139. The cup 3126 is configured to move from the undeployed to the deployed state, e.g., automatically. As depicted in FIGS. 31A and 31B, in the undeployed state, the cup 3126 remains close to the catheter body 3122. In some embodiments, the expandable cup 3126 may have a diameter in the deployed state of about 4 mm to about 20 mm (e.g., 4, 5, 6, 7, 8, 9, 10, 11, 12, 13, 14, 15, 16, 17, 18, 19, or 20 mm, inclusive).

As depicted in FIGS. 31C and 31D, in the deployed state, the cup 3126 expands, increasing its surface area such that the blood flow of the patient (e.g., left to right in the FIGS. 31A to 31D, but can be otherwise, i.e., the catheter 3120 may be inserted with the cup 3126 being orientated from left to right as shown or right to left) can be caught by the cup 3126 in its expanded configuration and drive the cup 3126 and thereby, the catheter 3120, toward the heart. Once at the heart, the cup 3126 can also facilitate navigation of the catheter through the heart chambers and into and/or through the pulmonary arteries. Once the catheter 3120 reaches the desired location within a branch of the pulmonary artery, the cup 3126 may automatically transition to the undeployed state (with a diameter of 10 mm or less, e.g., about 10 mm, about 9 mm, about 8 mm, about 7 mm, about 6 mm, about 5 mm, about 4 mm, about 3 mm, about 2 mm, about 1.33 mm, about 1 mm, or less, including any values and subranges in between) or compress to a smaller size, e.g., to occlude a branch of the pulmonary artery for measuring the PCWP. The material of the expandable cup 3126 allows for an automatic deployment and undeployment upon insertion of the catheter 3120 into a peripheral vein in the arm (e.g., antecubital fossa veins) through the vasculature up to the heart. It should be appreciated that while guide 3126 depicts a particular design, the guide 3126 can be combined with any of other designs described herein, or used in any of the systems described herein.

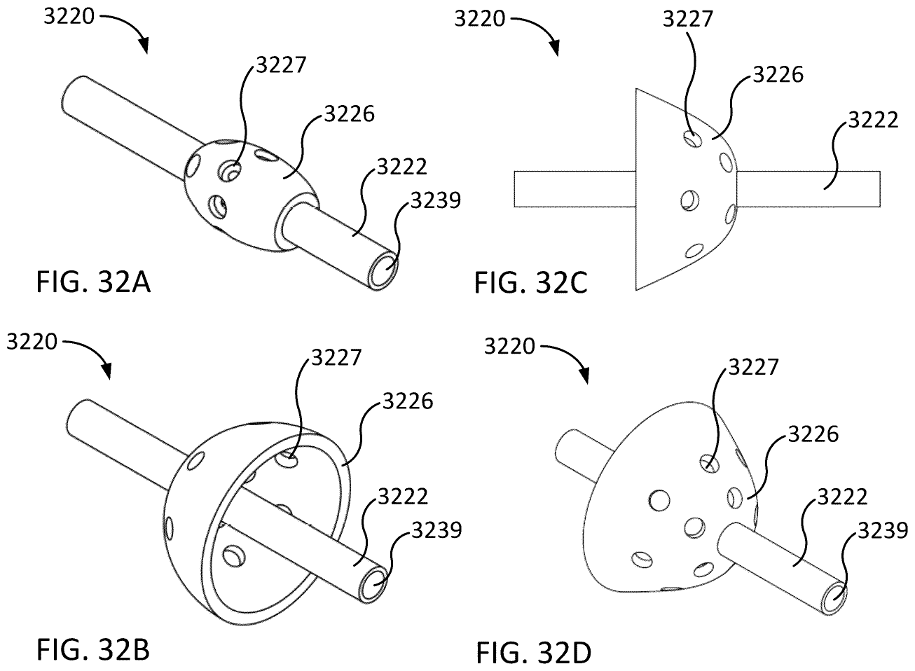

FIGS. 32A to 32D depict a catheter 3220 including a guide 3226 implemented as an expandable cup 3226 with holes 3227 (e.g., self-expanding gel, viscoelastic material, hydrogel, silicone, etc.), according to an embodiment. FIGS. 32A and 32B illustrate the undeployed and deployed states, respectively, of the guide 3226 in an isometric profile of the catheter 3220 including an expandable cup 3226 with holes 3227. FIGS. 32C and 32D illustrate another configuration of the undeployed state (e.g., a flipped configuration or position of the cup 3226), in a side profile and isometric profile, respectively, e.g., upon the insertion of a catheter body 3222 on which the cup 3226 is disposed into an IV catheter (sheath). In some embodiments, the flipped position of the cup 3226 (FIGS. 32C and 32D) upon insertion into a vessel can automatically transition to the deployed state (FIG. 32B). The guide 3226 is disposed on the catheter body 3222 that defines a lumen 3239. In some embodiments, the expandable cup 3226 with holes 3227 may have a diameter in the deployed state of about 4 mm to about 20 mm (e.g., 4, 5, 6, 7, 8, 9, 10, 11, 12, 13, 14, 15, 16, 17, 18, 19, or 20 mm, inclusive).

As depicted in FIG. 32A, in the undeployed state, the cup 3226 remains close to the catheter body 3222. As depicted in FIG. 32B, in the deployed state, the surface area of the guide 3220 can increase such that the blood flow of the patient (left to right in the FIGS. 32A to 32D, but can be from right to left, i.e., the catheter 3220 may be inserted with the cup 3226 being orientated from left to right as shown in FIGS. 32A and 32B, or right to left as shown in FIGS. 32C and 32D) can be caught by the expandable cup 3226 and drive the cup 3226 toward the heart. Once at the heart, the cup 3226 can also facilitate navigation of the catheter through the heart chambers and into and/or through the pulmonary arteries.

In some embodiments, the holes 3227 through the cup 3226 allow for blood flow through the cup 3226 to prevent blood stagnation. The number of holes 3227 on the surface can be greater or less than 12 depending on flow testing of the cup 3226. Once the catheter 3222 reaches the desired location within a branch of the pulmonary artery, the cup 3226 can automatically transition to the undeployed state (with a diameter of 10 mm or less, e.g., about 10 mm, about 9 mm, about 8 mm, about 7 mm, about 6 mm, about 5 mm, about 4 mm, about 3 mm, about 2 mm, about 1.33 mm, about 1 mm, or less, including any values and subranges in between) to occlude a branch of the pulmonary artery for measuring the PCWP. In some embodiments, the cup 3226 may be oriented from right to left (e.g., curving towards the left) as shown in FIGS. 32C and 32D during insertion and once it reaches a desired location, the cup 3226 may flip to be oriented from left to right (e.g., curving towards the right), as shown in FIGS. 32A and 32B. In some embodiments, the cup 3226 may have a hemispherical profile when curved from left to right, but may have a flared profile when curved from right to left as shown in FIGS. 32C and 32D. The material of the expandable cup allows for an automatic deployment and undeployment upon insertion of the catheter into a peripheral vein in the arm (e.g., antecubital fossa veins) through the vasculature up to the heart, as previously described. It should be appreciated that while guide 3226 depicts a particular design, the guide 3226 can be combined with any of other designs described herein, or used in any of the systems described herein.

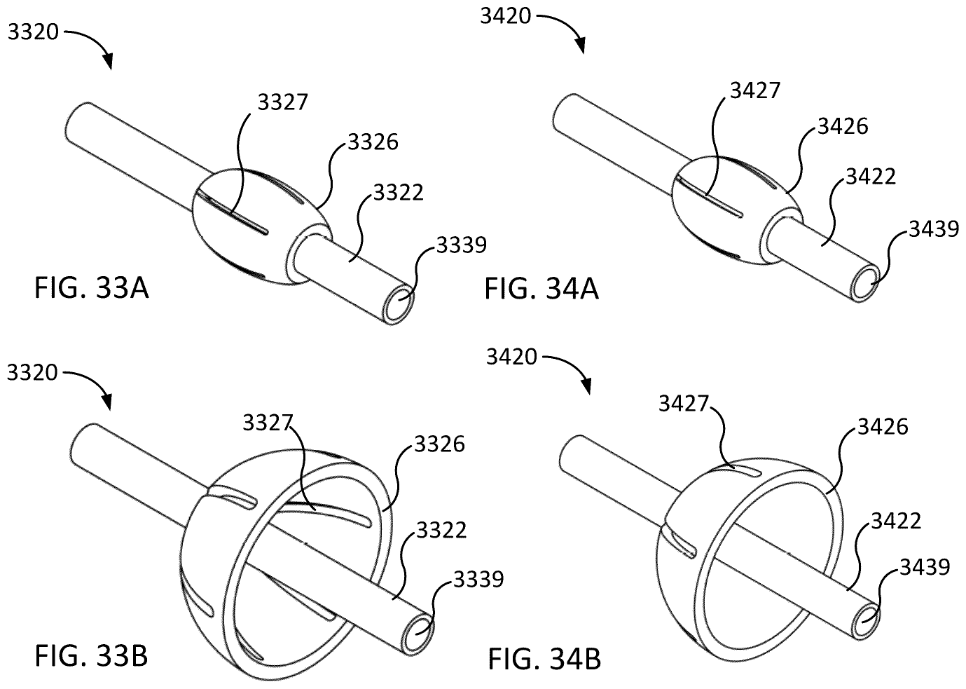

FIGS. 33A and 33B depict a catheter 3320 including a guide 3326 implemented as an expandable cup 3326 with cut-through slits 3327 (e.g., self-expanding gel, viscoelastic material, hydrogel, silicone, etc.), according to an embodiment. FIGS. 33A and 33B illustrate the undeployed and deployed states, respectively, of the guide 3326 in an isometric profile of the expandable cup design with cut-through slits. The guide 3326 is disposed on a catheter body 3322 that defines a lumen 3339. As depicted in FIG. 33A, in the undeployed state, the cup 3326 with cut-through slits 3327 (e.g., defined through the cup 3326) remains close to the catheter body 3322. The cut-through slits 3327 can be more spread out or be positioned closer to each other on the surface of the cup 3326 to allow for a better folding (e.g., collapse). The shape, size, spacing, etc. of the slits 3327 can also impact the total number of the cut-through slits 3327 used on the surface. In some embodiments, the expandable cup 3326 with cut-through slits 3327 may have a diameter in the deployed state of about 4 mm to about 20 mm (e.g., 4, 5, 6, 7, 8, 9, 10, 11, 12, 13, 14, 15, 16, 17, 18, 19, or 20 mm, inclusive).

As depicted in FIG. 33B, in the deployed state, the surface area of the cup 3326 will increase such that the blood flow of the patient (e.g., left to right in the FIGS. 33A and 33B, but can be otherwise, i.e., the guide 3320 may be inserted with the cup being orientated from left to right as shown in FIGS. 32A and 32B, or right to left) can be caught by the expandable cup 3326 and drive the cup 3326 toward the heart. Once at the heart, the cup 3326 can also facilitate navigation of the catheter through the heart chambers and into and/or through the pulmonary arteries. In some embodiments, the cut-through slits 3327 on the surface of the cup 3326 serve two purposes, e.g., allowing for the blood to flow through the cup 3326 to prevent blood stagnation and/or allowing for the cup 3326 to fold easier when automatically transitioning from the deployed state (FIG. 33B) back to the undeployed state (FIG. 33A). Once the catheter body 3322 reaches the desired location within a branch of the pulmonary artery, the cup 3326 can automatically transition to the undeployed state (with a diameter of 10 mm or less, e.g., about 10 mm, about 9 mm, about 8 mm, about 7 mm, about 6 mm, about 5 mm, about 4 mm, about 3 mm, about 2 mm, about 1.33 mm, about 1 mm, or less, including any values and subranges in between) or transition to a smaller size, e.g., to occlude a branch of the pulmonary artery for measuring the PCWP. The material of the expandable cup 3326 allows for an automatic deployment and undeployment upon insertion of the catheter body 3322 into a peripheral vein in the arm (e.g., antecubital fossa veins) through the vasculature up to the heart, as previously described. It should be appreciated that while guide 3326 depicts a particular design, the guide 3326 can be combined with any of other designs described herein, or used in any of the systems described herein.

FIGS. 34A and 34B depict a catheter 3420 including a guide 3426 implemented as an expandable cup 3426 with engraved slits 3427 (e.g., self-expanding gel, viscoelastic material, hydrogel, silicone, etc.), according to an embodiment. FIGS. 34A and 34B illustrate the undeployed and deployed states, respectively, of the guide 3426 in an isometric profile of the expandable cup 3426 design with engraved slits 3427 (or other depressions) that are formed on a surface of the cup 3426 but do not extend therethrough. The guide 3426 is disposed on a catheter body 3422 that defines a lumen 3439. As depicted in FIG. 34A, in the undeployed state, the cup 3426 with engraved slits 3427 remains close to the catheter body 3422. In some embodiments, the expandable cup 3426 with engraved slits 3427 may have a diameter in the deployed state of about 4 mm to about 20 mm (e.g., 4, 5, 6, 7, 8, 9, 10, 11, 12, 13, 14, 15, 16, 17, 18, 19, or 20 mm, inclusive).

The engraved slits 3427 can be more spread out or be positioned closer to each other on the surface of the cup 3426 to allow for easier folding (e.g., collapse), which can impact the total number of the engraved slits 3427 formed on the surface. It should be appreciated that while the engraved slits 3427 are depicted as being on the outer surface of the cup 3426, the engraved slits 3427 may be on the inner surface of the cup 3426, or the engraved slits 3427 may be on both surfaces. As depicted in FIG. 34B, in the deployed state, the surface area of the guide 3420 can increase such that the blood flow of the patient (e.g., left to right in the FIGS. 34A to 34B, but can be otherwise, as previously described) can be caught by the expandable cup 3426 and drive the cup 3426 toward the heart. Once at the heart, the cup 3426 can also facilitate navigation of the catheter through the heart chambers and into and/or through the pulmonary arteries.

In some embodiments, the engraved slits 3427 on the surface of the cup 3426 allow for the blood to flow around the cup 3426 (e.g., to prevent blood stagnation) and/or help the cup 3426 to fold easier when automatically transitioning from the deployed state (FIG. 34B) back to the undeployed state (FIG. 34A). Once the catheter body 3422 reaches the desired location within a branch of the pulmonary artery, the cup 3426, can automatically transition to the undeployed state (with a diameter of 10 mm or less, e.g., about 10 mm, about 9 mm, about 8 mm, about 7 mm, about 6 mm, about 5 mm, about 4 mm, about 3 mm, about 2 mm, about 1.33 mm, about 1 mm, or less, including any values and subranges in between) or transition to a smaller size, e.g., to occlude a branch of the pulmonary artery for measuring the PCWP. In some embodiments, the engraved slits 3427 on the cup 3426 surface form a closed profile that completely blocks blood flow through the cup 3426, in both undeployed and deployed states, and allows for a better navigation to the heart by catching on the blood flow. The material of the expandable cup 3426 allows for an automatic deployment and undeployment upon insertion of the catheter body 3422 into a peripheral vein in the arm (e.g., antecubital fossa veins) through the vasculature up to the heart, as previously described. It should be appreciated that while guide 3426 depicts a particular design, the guide 3426 can be combined with any of other designs described herein, or used in any of the systems described herein.

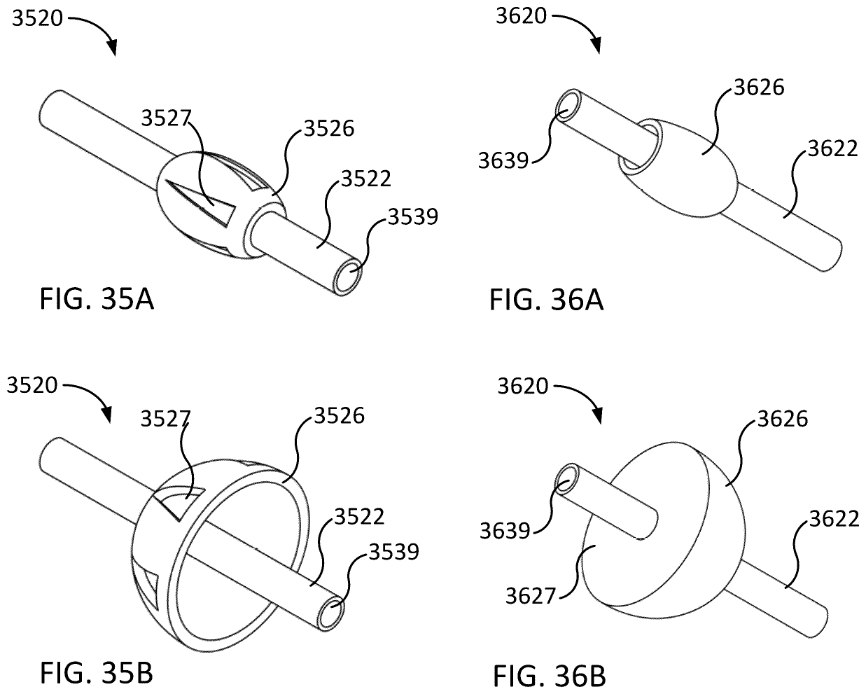

FIGS. 35A and 35B depict a catheter 3520 including a guide implemented as an expandable cup 3526 with engraved triangles 3527 (e.g., self-expanding gel, viscoelastic material, hydrogel, silicone, etc.), according to an embodiment. FIGS. 35A and 35B illustrate the undeployed and deployed states, respectively, of the guide 3526 in an isometric profile of the expandable cup 3526 design with engraved triangles 3527. The guide 3526 is disposed on a catheter body 3522 that defines a lumen 3539. As depicted in FIG. 35A, in the undeployed state, the cup 3526 with engraved triangles 3527 remains close to the catheter body 3522. The engraved triangles 3527 can be more spread out or be positioned closer to each other on the surface of the cup 3526 to allow for easier folding (e.g., collapse), which can impact the total number of the engraved triangles 3527 used on the surface. In some embodiments, a base of each of the engraved triangles 3527 is positioned proximate to the expanding end of longitudinal end of the cup 3526 that may facilitate folding and unfolding of the cup 3526. It should be appreciated that while the engraved triangles 3527 are depicted as being on the outer surface of the cup 3526, the engraved triangles 3527 may be on the inner surface of the cup 3526, or the engraved triangles 3527 may be on both surfaces. While FIGS. 35A and 35B show the cup 3526 as having engraved triangles 3527, in some embodiments, engraved features formed on an outer surface of the cup 3526 can have any suitable shape, for example, square, rectangular, circular, oval, polygonal, etc. In some embodiments, the expandable cup 3526 with engraved triangles 3527 may have a diameter in the deployed state of about 4 mm to about 20 mm (e.g., 4, 5, 6, 7, 8, 9, 10, 11, 12, 13, 14, 15, 16, 17, 18, 19, or 20 mm, inclusive).

As depicted in FIG. 35B, in the deployed state, the surface area of the guide 3526 can increase such that the blood flow of the patient (e.g., left to right in the FIGS. 35A and 35B, but can be otherwise, as previously described) can be caught by the expandable cup 3526 and drive the cup 3526 toward the heart. Once at the heart, the cup 3526 can also facilitate navigation of the catheter through the heart chambers and into and/or through the pulmonary arteries. In some embodiments, the engraved triangles 3527 on the surface of the cup 3526 allow for the blood to flow around the cup 3526 and help the cup 3526 to fold easier (e.g., due to a similar shape to the collapsible umbrella structures) when automatically transitioning from the deployed state (FIG. 35B) back to the undeployed state (FIG. 35A).

Once the catheter body 3522 reaches the desired location within a branch of the pulmonary artery, the cup 3526 can automatically transition to the undeployed state (with a diameter of 10 mm or less, e.g., about 10 mm, about 9 mm, about 8 mm, about 7 mm, about 6 mm, about 5 mm, about 4 mm, about 3 mm, about 2 mm, about 1.33 mm, about 1 mm, or less, including any values and subranges in between) or transition to a smaller size, e.g., to occlude a branch of the pulmonary artery for measuring the PCWP. In some embodiments, the engraved triangles 3527 on the cup 3526 surface form a closed profile that completely blocks blood flow through the cup 3526, in both undeployed and deployed states, and allows for a better navigation to the heart by catching on the blood flow. The material of the expandable cup 3526 allows for an automatic deployment and undeployment upon insertion of the catheter body 3522 into a peripheral vein in the arm (e.g., antecubital fossa veins) through the vasculature up to the heart, as previously described. It should be appreciated that while guide 3526 depicts a particular design, the guide 3526 can be combined with any of other designs described herein, or used in any of the systems described herein.

FIGS. 36A and 36B depict a catheter 3620 including a guide implemented as an expandable cup 3626 with a closed flat surface 3627 (e.g., self-expanding gel, viscoelastic material, hydrogel, silicone, etc.), according to an embodiment. FIGS. 36A and 36B illustrate the undeployed and deployed states, respectively, of the guide 3626 in an isometric profile of the expandable cup 3626 design with the closed flat surface 3627. The guide 3626 is disposed on a catheter body 3622 that defines a lumen 3639. In some embodiments, the expandable cup 3626 with a closed flat surface 3627 may have a diameter in the deployed state of about 4 mm to about 20 mm (e.g., 4, 5, 6, 7, 8, 9, 10, 11, 12, 13, 14, 15, 16, 17, 18, 19, or 20 mm, inclusive).

As depicted in FIG. 36A, in the undeployed state, the cup 3626 with the closed flat surface 3627 remains close to the catheter body 3622. In some embodiments, engraved structures (e.g., slits, triangles, etc.) can be defined on the flat surface 3627 to allow for an easier folding. As depicted in FIG. 36B, in the deployed state, the surface area of the guide 3626 can increase such that the blood flow of the patient (e.g., left to right in the FIGS. 36A and 36B, but can be otherwise, as previously described) can be caught by the expandable cup 3626 and drive the cup 3626 towards the heart. In some embodiments, the blood flow pushes the flat surface 3627 of the cup 3626 and allows for an easier catching on the flow and navigation through the vasculature. Once the catheter body 3622 has reached the target location within a branch of the pulmonary artery, the cup 3626 with the flat surface 3627 can automatically transition to the undeployed state (e.g., with a diameter of 10 mm or less (e.g., about 10 mm, about 9 mm, about 8 mm, about 7 mm, about 6 mm, about 5 mm, about 4 mm, about 3 mm, about 2 mm, about 1.33 mm, about 1 mm, or less, including any values and subranges in between) or another smaller state, e.g., to occlude a branch of the pulmonary artery for measuring the PCWP. The material of the expandable cup 3626 allows for an automatic deployment and undeployment upon insertion of the catheter body 3622 into a peripheral vein in the arm (e.g., antecubital fossa veins) through the vasculature up to the heart, as previously described. It should be appreciated that while guide 3626 depicts a particular design, the guide 3626 can be combined with any of other designs described herein, or used in any of the systems described herein.

In some embodiments, the catheters described herein can be stored and deployed using other configurations, such as a fishing rod structure (e.g., spool or reel dispenser in a fishing rod), a kite structure (e.g., spool or line winder), a garden hose structure (e.g., hose reel), or a retractable cord structure. More information about retractable cord structure can be found, e.g., in U.S. Pat. No. 6,616,080, entitled “Retractable cord device,” issued Sep. 9, 2003, which is incorporated by reference in its entirety.

While various embodiments have been described and illustrated herein, a variety of other means and/or structures for performing the function and/or obtaining the results and/or one or more of the advantages described herein, and each of such variations and/or modifications are possible. More generally, all parameters, dimensions, materials, and configurations described herein are meant to be examples and that the actual parameters, dimensions, materials, and/or configurations will depend upon the specific application or applications for which the disclosure is used. It is to be understood that the foregoing embodiments are presented by way of example only and that other embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

Some embodiments described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

What is claimed is:

1. An apparatus, comprising:
an elongate body including a proximal portion and a distal portion, the elongate body defining a lumen;
a set of sensors disposed on the distal portion of the elongate body;
an electrical connector coupled to the proximal portion of the elongate body, the electrical connector coupled to the set of sensors via an electrical wire extending through the elongate body; and
a guide member disposed on the distal portion near the set of sensors, the guide member transitionable between an undeployed configuration and a deployed configuration, the guide member in the deployed configuration configured to guide movement of the distal portion of the elongate body through a vascular system of a subject using blood flow,
the elongate body having a diameter less than or equal to 8 millimeters (mm).

2. The apparatus of claim 1, wherein the guide member is a balloon, the apparatus further comprising:
an inflation port coupled to the proximal portion of the elongate body, the inflation port configured to deliver a fluid via the lumen of the elongate body to transition the balloon from the undeployed configuration to the deployed configuration.

3. The apparatus of claim 2, wherein the balloon defines an internal volume, the balloon including a self-expandable structure disposed within the interior volume of the balloon and configured to cause the balloon to selectively transition between the undeployed configuration and the deployed configuration.

4. The apparatus of claim 2, wherein the balloon is solid, the balloon being formed from a self-expandable material configured to selectively expand and contract within the vascular system to move selectively transition the balloon between the undeployed configuration and the deployed configuration.

5. The apparatus of claim 4, wherein the balloon includes at least one engraved slit defined on an outer surface of the balloon.

6. The apparatus of claim 1, wherein the guide member is a sail, the sail being configured to transition from the undeployed configuration to the deployed configuration in response to being disposed within the vascular system.

7. The apparatus of claim 6, wherein the sail includes at least one petal extending radially outward from the elongate body, at least a portion of the at least one petal configured to selectively move towards the elongated body or away from the elongated to transition the sail between the undeployed state and the deployed state, respectively.

8. The apparatus of claim 7, wherein:
the sail includes a plurality of petals, and webbing disposed between each of the plurality of petals.

9. The apparatus of claim 6, further including:
a deployment mechanism configured to transition the guide member from the undeployed configuration to the deployed configuration.

10. The apparatus of claim 6, wherein the deployment mechanism is a slider that is coupled via a shaft to the sail,
the slider configured to move to slide the shaft to transition the guide member from the undeployed configuration to the deployed configuration,
the shaft slidably disposed within the lumen of the elongate body.

11. The apparatus of claim 1, wherein the guide member includes a cup, a first end of the cup coupled to an outer surface of the elongate body, and a second end configured to move selectively towards the elongate body or away from the elongate body to transition the guide member between the undeployed configuration and the deployed configuration, respectively.

12. The apparatus of claim 11, wherein the cup includes a at least one opening defined through the cup.

13. The apparatus of claim 11, wherein the cup includes at least one engraved slit defined on a surface of the cup.

14. The apparatus of claim 13, wherein the engraved slit defines a longitudinal shape or a triangular shape.

15. The apparatus of claim 11, wherein the cup includes a flat surface positioned to face a direction of blood flow in the deployed configuration.

16. A system, comprising:
a catheter including:
an elongate body including a proximal portion and a distal portion, the elongate body defining a lumen;
a set of sensors disposed on the distal portion of the elongate body; and
a guide member disposed on the distal portion near the set of sensors, the guide member transitionable between an undeployed configuration and a deployed configuration, the guide member in the deployed configuration configured to guide movement of the distal portion of the elongate body through a vascular system of a subject using blood flow; and
a housing including:
a sterile chamber configured to house a portion of the elongate body of the catheter; and
a connector configured to couple to a hub of an intravenous catheter such that the distal portion of the catheter can be advanced out of the sterile chamber and through a lumen of the intravenous catheter and into the vascular system.

17. The system of claim 16, wherein the portion of the elongate body of the catheter is wound in a spool within the sterile chamber prior to being advanced out of the sterile chamber.

18. The system of claim 17, further including a motor coupled to spool, the motor configured to selectively rotate the spool for advancing the catheter out of or into the sterile chamber.

19. The system of claim 16, wherein the portion elongate body is wound around a first pole and a second pole laterally spaced apart from the first pole such that portion of the elongate body forms a coil having a bow shape.

20. The system of claim 16, further including at least two feeders, a portion of the catheter disposed between the at least two feeders, the at least two feeders configured to rotate in opposite directions to selectively draw the portion of the elongate body out of the sterile chamber or retract the portion into the sterile chamber.

21. The system of claim 20, further including torquing assembly coupled to the at least two feeders, the torquing assembly configured to cause the at least two feeders to displace relative to a longitudinal axis of the catheter to cause the portion of the catheter disposed between the at least two feeders to bend or rotate so as to exert a torque on the catheter.

22. The system of claim 16, wherein the housing further includes a set of ports for coupling at least one of a compute device or a fluid source outside of a sterile field to the catheter while maintaining the catheter within the sterile field.

23. A method, comprising:
coupling a connector of a housing containing a right heart catheter to a hub of an intravenous catheter;
advancing a distal portion of the right heart catheter through a lumen of the intravenous catheter and into a peripheral vein of a subject;
deploying a guide of the right heart catheter such that the guide enables the distal portion of the right heart catheter to be carried by blood flow through a vascular system of the subject and to a pulmonary vessel of a heart of the subject; and
measuring one or more physiological conditions of the subject via a set of sensors of the right heart catheter when the distal portion of the right heart catheter is disposed in the pulmonary vessel.

24. The method of claim 23, wherein the peripheral vein is an antecubital vein.

25. The method of claim 23, further comprising:
capturing, while the distal portion of the right heart catheter is being carried through the vascular system, pressure data via a pressure sensor of the right heart catheter, the pressure data indicative of a location of the distal portion of the right heart catheter.

26. The method of claim 23, further comprising:
transitioning the guide of the right heart catheter into a sample collection configuration after the distal portion of the right heart catheter is disposed in the pulmonary vessel, the guide in the sample collection configuration configured to capture and retain a volume of blood within the pulmonary vessel.

27. The method of claim 23, further comprising:
occluding the pulmonary vessel prior to measuring the one or more physiological conditions of the subject.

28. An apparatus, comprising:
an elongate body including a proximal portion and a distal portion, the distal portion of the elongate body disposable within a subject;
a set of sensors disposed on the distal portion of the elongate body, the set of sensors configured to measure one or more physiological conditions of the subject when the distal portion of the elongate body is disposed within the subject, the set of sensors configured to wirelessly transmit data indicative of the one or more physiological to at least one of a communication element or a compute device;
a guide member disposed on the distal portion near the set of sensors, the guide member transitionable between an undeployed configuration and a deployed configuration, the guide member in the deployed configuration configured to guide movement of the distal portion of the elongate body through a vascular system of the subject using blood flow,
the elongate body having a diameter less than or equal to 8 millimeters (mm).
